(19) United States
(12) Patent Application Publication
(10) Pub. No.: US 2020/0399578 A1
(43) Pub. Date: Dec. 24, 2020

(54) Devices, Methods, and Systems for Electroporation

(71) Applicant: CyteQuest, Inc., Ithaca, NY (US)

(72) Inventors: Tom Corso, Groton, NY (US); Harold G. Craighead, Ithaca, NY (US)

(21) Appl. No.: 16/876,718

(22) Filed: May 18, 2020

Related U.S. Application Data
(60) Provisional application No. 62/848,944, filed on May 16, 2019.

(51) Int. Cl. C12M 1/42; C12N 15/87

(52) U.S. Cl. CPC C12M 35/02; C12N 15/87

(57) ABSTRACT

Disclosed are a system, device, and method for the electroporation of cells. Systems, devices and methods for electroporation of living cells and the introduction of selected molecules into the cells utilizes a fluidic system where living cells and biologically active molecules flow through a channel that exposes them to electric fields, causing the molecules to be transferred across the cell membrane. The methods are particularly well suited for the introduction of DNA, RNA, drug compounds, and other biologically active molecules into living cells for use in cell-based therapies.

### CROSS-REFERENCE TO RELATED APPLICATION

This application claims the benefit of the filing date of U.S. Provisional Patent Application Ser. No. 62/848,944, filed on May 16, 2019; which is hereby incorporated by reference in its entirety.

### BACKGROUND

In medicine and biomedical research, there is often a need to insert biologically active molecules into selected living cells. These molecules could be drugs to treat specific diseases, but an important application is the insertion of nucleic acid molecules such as DNA and RNA, which is typically called transfection or transformation. The inserted nucleic acid molecules can serve as a vaccine, can enable the cellular production of specific proteins, or can be used to reprogram the human immune system cells to attack tumors or disease-causing cells. In such applications, it is critical to insert sufficient DNA or RNA or nucleic acid protein complexes into a cell without doing damage that could kill the cell. Control of the process is important, and process parameters generally differ for different types of cells. It is also important for the cell transformation process to operate reliably, reproducibly, and with sufficient speed to transform the required number of cells in as short a time as possible.

A method known as electroporation, or electropermeabilization, has been used for decades as an approach to electrically open pores in cell membranes to allow the passage of molecules into the cells. In this method, electric fields are created by applying high voltage electrical pulses to electrodes inserted in a liquid container containing cells suspended in a liquid solution that contains the molecules to be inserted into the cells. The applied high-voltage pulses create transient pores in the cell membrane that allow molecules to pass into the cells. However, the open pores also allow the cell contents to escape and undesired molecules to diffuse into the cell with negative consequences for the health of the cell. The pulse voltage, number of pulses, and pulse duration are among the parameters empirically varied to optimize the efficiency of molecular insertion and cell survival. However, current devices are limited in that cells and molecules are exposed to a large range of electric fields, often causing biologically active molecules to not transfer efficiently and/or many cells to be damaged or killed during electroporation. Current devices also lack control of process parameters; therefore, the process cannot be controlled and optimized for various cell types and biologically active molecules. Furthermore, current devices have limited throughput. These drawbacks have limited the widespread application of this method.

Some improvement in throughput has been made by flowing a solution with living cells and biologically active molecules through the container with electrodes. For example, a publication by Choi et al. (2010) proposed a high-throughput microelectroporation device for introducing chimeric antigen receptor to human T cells to redirect their specificity. In addition, U.S. Pat. Nos. 4,752,586; 5,612,207; 6,074,605; and 6,090,617 (each incorporated by reference) refer to electroporation with flow for processing large number of cells. These devices introduce flow to fill and empty the electroporation chamber, but the efficiency of molecular transformation as well as the potential lysing of the cells remains a problem.

U.S. Patent Application Publication 2014/0066836 (incorporated by reference) discloses an electroporation device that includes movable electrodes to achieve a more specific spatial configuration between the electrodes and the cells. However, the cells reside in a bulk solution in the device or in vivo. Thus, the quantity of cells that are exposed to the precise field strength is limited.

There are also additional practical limitations of the current electroporation methods. For example, high voltages are required, and it is often necessary to pulse the voltage to allow the cells to recover in between voltage exposure. Also, current devices permit cells to be porated only in a single, homogenous fluid environment. In addition, current devices preclude the ability to monitor the cell motion and electroporation process optically or electrically while it is occurring.

Currently, the art lacks a system, method, and device for the introduction of biologically active molecules into flowing living cells by electroporation in a manner that allows control of various parameters that affect the efficiency of electroporation.

### SUMMARY

In accord with one aspect of the present disclosure there is provided a device capable of inserting a biologically active molecule into living cells including a planar fluid channel including at least one fluid input and a fluid output configured to allow a fluid flow including living cells and biologically active molecules through the channel; and electrodes on opposite sides of the fluid channel to which electrical potentials can be applied to form a time-varying electric field directed across the fluid channel, wherein different electrodes can be maintained at different electrical potentials to provide a spatially-varying electric field within the channel. In on embodiment, the distance between the first and second electrodes enables the cells to pass through the space between the electrodes in a single layer so a living cell in the fluid flow is maintained in a similar position as other living cells in the fluid flow as they pass through the planar channel in a manner that prevents one living cell from shielding another living cell from the applied electric field, wherein the strength of the electric field to which the living cell is exposed is sufficient to form pores within the membrane of the living cell through which the biologically active molecule can traverse the cell membrane, but not lyse the living cell. Furthermore, the electric field can be controlled in time and in position throughout the channel to create the most advantageous conditions for device throughput, cell viability and transfection efficiency. The disclosed device can be incorporated in a variety of systems for manufacturing cells for therapeutic uses and used for research in developing new cell-based therapeutic approaches.

In accord with another aspect of the present disclosure there is provided a device capable of inserting a biologically active molecule into a living cell including a fluid planar channel including a fluid channel including at least two fluid inputs and a fluid output configured to independently control the composition and fluid flow rates in the fluid streams comprising living cells, biologically active molecules and chemical solutions through the channel; and electrodes on opposite sides of the fluid channel to which electrical potentials can be applied to form an time-varying and spatially-varying electric field directed across the fluid channel, wherein the dimensions of the fluid channel and the two or more laminar sheath fluid streams are sufficient to force the cells to pass through the space between the electrodes predominantly in a single layer so a living cell in the fluid flow is maintained in a similar position as other living cells in the fluid flow as they pass through the electric field between the first and second electrodes in a manner that prevents one living cell from shielding another living cell from the applied electric field, wherein the strength of the electric field to which the living cell is exposed is sufficient to form pores within the membrane of the living cell through which the biologically active molecule can traverse the cell membrane, but not lyse the living cell.

In accord with another aspect of the present disclosure there are provided designs for fluid channel devices and method for manufacturing planar fluid channel devices by efficient methods such as injection molding and bonding. Also presented are designs for coupling cylindrical tubes or pipes conveying fluid from pumps and fluid control systems into the multi-channel devices.

In accord with another aspect of the present disclosure there are provided designs for planar microfluidic devices and systems that integrate devices for cell sorting or cell separation up-stream from the cell electroporation systems. In this embodiment the specific cell types desired to be electroplated can be selected, thereby improving the efficiency of the process, or removing undesired cells form the process.

In accord with another aspect of the present disclosure are systems and methods for utilizing the planar multiple flow devices for the modification of cells for medical applications. In one embodiment, cells from human or animal blood are modified by controlled variation of fluid flow and electrical parameters to efficiently determine the optimum conditions for a particular combination of biological molecules for efficiency of cell modification and cell viability. In an embodiment, T cells from patient blood are modified by optimized conditions for rapidly for therapeutic administration.

The present invention involves, in some embodiments, electrically activating electrodes in a flow-based electroporation system with periodic time-dependent voltage characteristics to improve the effectiveness of cell electroporation and to mitigate issues of surface charge and reactions on electrodes. This aspect of the invention differs explicitly from the standard practice of exposing batches of cells to one or a few voltage pulses. In some embodiments, a prescribed voltage waveform is utilized in a continuous manner so that cells that are moving in a flowing liquid in a cannel will experience different voltages at different times.

In some aspects, electroporation devices include a first-side support having at least one first-side electrode disposed at a region of an inner surface of the first-side support; a second-side support having at least one second-side electrode disposed at a region of an inner surface of the second-side support, in which the at least one second-side electrode is a counterpart electrode of the at least one first-side electrode; and a fluid channel, having at least a portion of it between the first-side support and the second-side support, and having at least one fluid input and at least one fluid output, in which the fluid channel allows fluid to flow continuously in at least one fluid stream toward the at least one fluid output, in which the at least one first-side electrode and the at least one second-side electrode are positioned in the electroporation device to allow modulating the electric field as a predetermined function of time and position in at least one part (e.g., at least two parts, at least three parts, at least four parts, at least five parts, at least six parts) of the fluid channel between the first-side support and the second-side support.

In some embodiments, the shortest distance between any electrode on the first-side support and any of its counterpart electrodes on the second-side support is at most 1 millimeters (e.g., at most 50, 100, 150, 200, 250, 300, 350, 400, 450, 500, 550, 600, 650, 700, 750, 800, 850, 900, 950, or 1000 micrometers). In other embodiments, this distance can be further increased (e.g., to 2 millimeters or more), with an accompanying increase in the voltage (e.g., to 500 V for a distance of 10 millimeters) to effect a similar electric field within the fluid channel.

In some embodiments, the electroporation device further includes at least one voltage supplier, in which the at least one first-side electrode and the counterpart electrode of the at least one first-side electrode are connected to the voltage supplier independently from any other electrodes of the electroporation device, and in which the voltage supplier allows modulating the electric field as a function of time in the at least one part (e.g., at least two parts, at least three parts, at least four parts, at least five parts, at least six parts) of the fluid channel. In some embodiments, the electroporation device has a plurality of first-side electrodes including the at least one first-side electrode, and including a plurality of second-side electrodes including the at least one second-side electrode, in which at least two sets of counterpart electrodes operate independently from each other. In certain embodiments, at least three sets of counterpart electrodes operate independently from each other. In some embodiments, the electroporation device further includes one or more additional voltage suppliers, in which each voltage supplier is connected to a different set of counterpart electrodes in the electroporation device. In certain embodiments, the voltage suppliers allow forming an electric field as a function of time and position within the fluid channel that maximizes an outcome function that positively correlates with cell transfection efficiency and negatively correlates with cell mortality. In certain embodiments, the voltage suppliers allow forming an electric field as a function of time and position within the fluid channel that maximizes an outcome function that positively correlates with electrode durability. In certain embodiments, the voltage suppliers allow independently controlling any two or more of the following: (a) opening pores in cells in a fluid in the fluid channel; (b) driving molecules into cells in a fluid in the fluid channel; (c) measuring an electrical property of a fluid in the fluid channel; (d) concentrating molecules at a part of the fluid channel; (e) moving cells in a fluid in the fluid channel to a part of the fluid channel; and (f) rotating cells in a fluid in the fluid channel.

In some embodiments, the voltage supplier provides a voltage that has a periodic waveform. In certain embodiments, the periodic waveform is a sinusoidal function of time, in which the sinusoidal function has an absolute amplitude from zero that is at most 50 Volts, a frequency that is at least 10 Hz and at most 100 kHz, and a phase that is at least 0 and at most 2n. In some embodiments, the periodic waveform has a first frequency and a second frequency different from the first frequency. In some embodiments, the periodic waveform is a Fourier series. In some embodiments, the periodic waveform is a square waveform having a voltage amplitude of at least 0.1 V and at most 100 V, and a frequency of at least 100 Hz and at most 1 THz. In some embodiments, the square waveform is bipolar. In some embodiments, the square waveform further includes a direct current component of at most ±10 V.

In certain embodiments, the inner surface of the first-side support and the inner surface of the second-side support are substantially planar. In some embodiments, the portion of the fluid channel has a first surface facing the first-side and a second surface facing the second side, and in which both the first surface and the second surface are substantially planar. In some embodiments, the modulating the electric field as a predetermined function of time and position dynamically controls the electric field without requiring discrete electrical pulses. In some embodiments, the fluid channel includes at least two fluid inputs. In some embodiments, the fluid channel allows a laminar flow of fluids from the at least two fluid inputs.

In some embodiments, the average width of the portion of the fluid channel between the first-side support and the second-side support, as measured along a direction substantially parallel to the first-side support and the second-side support and perpendicular to the flow of the at least one fluid stream, is at least 10-fold and at most 1000-fold greater (e.g., 20-, 30-, 40-, 50-, 60-, 70-, 80-, 90-, 100-, 150-, 200-, 250-, 300-, 350-, 400-, 450-, 500-, 550-, 600-, 650-, 700-, 750-, 800-, 850-, 900-, 950-fold greater) than the average height of the portion of the fluid channel between the first-side support and the second-side support, as measured along a direction substantially perpendicular to the first-side support and the second-side support and parallel to the flow of the at least one fluid stream.

In some aspects, electroporation devices include a first-side support having at least one first-side electrode disposed at a region of an inner surface of the first-side support; a second-side support having at least one second-side electrode disposed at a region of an inner surface of the second-side support, in which the at least one second-side electrode is a counterpart electrode of the at least one first-side electrode; and a fluid channel, having at least a portion of it between the first-side support and the second-side support, and having at least two fluid inputs and at least one fluid output, in which the fluid channel allows fluid to flow continuously in at least two fluid streams toward the at least one fluid output, in which the fluid channel allows modulating the flow rate, chemical composition, or both the flow rate and chemical composition in the at least two fluid streams as a predetermined function of time, position, or both time and position.

In some embodiments of such electroporation devices, the shortest distance between any electrode on the first-side support and any of its counterpart electrodes on the second-side support is at most 1 millimeters (e.g., at most 50, 100, 150, 200, 250, 300, 350, 400, 450, 500, 550, 600, 650, 700, 750, 800, 850, 900, 950, or 1000 micrometers). In other embodiments, this distance can be further increased (e.g., to 2 millimeters or more), with an accompanying increase in the voltage (e.g., to 500 V for a distance of 10 millimeters) to effect a similar electric field within the fluid channel.

In some embodiments, the electroporation devices further include at least one fluid supplier, in which the fluid supplier allows modulating the flow rate and chemical composition in one of the at least two streams as a predetermined function of time, position, or both time and position independently from any other fluid stream within the fluid channel. In some embodiments, the electroporation devices further include one or more additional fluid suppliers, in which each fluid supplier is connected to a different fluid input in the fluid channel. In certain embodiments, the fluid suppliers allow modulating the flow rate and chemical composition in separate fluid streams as a function of time and position within the fluid channel to maximize the time a cell in a fluid stream is in its optimal medium. In certain embodiments, the fluid suppliers allow modulating the flow rate and chemical composition in separate fluid streams as a function of time and position within the fluid channel to minimize the time a cell in a fluid stream is in an electroporation medium. In certain embodiments, the fluid channel has at least three fluid inputs allowing fluid to flow continuously in at least three fluid streams toward the at least one fluid output. In certain embodiments, the inner surface of the first-side support and the inner surface of the second-side support are substantially planar. In certain embodiments, the portion of the fluid channel has a first surface facing the first-side and a second surface facing the second side, and in which both the first surface and the second surface are substantially planar. In certain embodiments, the modulating the flow rate, chemical composition, or both the flow rate and chemical composition in the at least two fluid streams as a predetermined function of time, position, or both time and position dynamically controls the electroporation process.

In some embodiments of such electroporation devices, the average width of the portion of the fluid channel between the first-side support and the second-side support, as measured along a direction substantially parallel to the first-side support and the second-side support and perpendicular to the flow of the at least one fluid stream, is at least 10-fold and at most 1000-fold greater (e.g., 20-, 30-, 40-, 50-, 60-, 70-, 80-, 90-, 100-, 150-, 200-, 250-, 300-, 350-, 400-, 450-, 500-, 550-, 600-, 650-, 700-, 750-, 800-, 850-, 900-, 950-fold greater) than the average height of the portion of the fluid channel between the first-side support and the second-side support, as measured along a direction substantially perpendicular to the first-side support and the second-side support and parallel to the flow at least one of the at least two fluid streams.

In certain aspects, systems include an electroporation device and a fluid-delivery apparatus coupled to the electroporation device. The electroporation device, in various embodiments, has the features described above and elsewhere.

In some embodiments of the system, the fluid-delivery apparatus includes a flow-rate control module. In some embodiments, the fluid-delivery apparatus includes a temperature control module. In some embodiments, the system further includes a fluid interface that couples the fluid-delivery apparatus to the electroporation device. In certain embodiments of the system, the electroporation device further includes at least one voltage control module. In some embodiments, the system further includes an electrical or optical monitoring module coupled to the electroporation device. In certain embodiments, the system further includes a cell processing module coupled to the electroporation device. In some embodiments, the cell processing module is upstream from the electroporation device. In some embodiments, the cell processing module allows cell sorting, selection, labeling, analysis, or a combination thereof. In certain embodiments, the cell processing module includes a fluorescence-activated cell sorting component. In some embodiments, the cell processing module includes a magnetic field source that allows magnetic bead separation. In certain embodiments, the system further includes an apheresis bag upstream of the cell processing module. In some embodiments, the system further includes a cell collection reservoir downstream of the electroporation device.

In some aspects, methods of forming an electroporation device include molding a material to form at least two support blocks, at least one support block having at least one input opening, and at least one support block having at least one output opening; attaching at least one electrode to each of the two support blocks to obtain two supports; and laminating the at least two supports together to form an electroporation device, in which the electroporation device has a channel between the two supports.

In some embodiments of such methods of forming an electroporation device, the molding includes injection molding. In some embodiments of methods of forming an electroporation device, attaching includes thermal bonding. In certain embodiments of methods of forming an electroporation device, the material is optically transparent.

In certain aspects, methods of electroporating a molecule into a cell include flowing a fluid at a flow rate through a fluid channel in an electroporation device, in which the fluid includes at least one cell and at least one molecule, and in which the fluid channel has at least one dimension that is at most 10 millimeters (e.g., at most 50, 100, 150, 200, 250, 300, 350, 400, 450, 500, 550, 600, 650, 700, 750, 800, 850, 900, 950, 1000, 1500, 2000, 2500, 3000, 3500, 4000, 4500, 5000, 5500, 6000, 6500, 7000, 7500, 8000, 8500, 9000, or 9500 micrometers); and applying to the fluid an electric field that varies as a predetermined function of time and position in at least one part (e.g., at least two parts, at least three parts, at least four parts, at least five parts, at least six parts) of the fluid channel.

In some embodiments of such methods, the fluid includes at least two streams, and in which the at least one cell is in one of the at least two streams and the at least one molecule is in another stream among the at least two streams. In some embodiments, the at least two streams have different chemical compositions. In certain embodiments, the at least one cell is a plurality of human T cells. In some embodiments, the at least one molecule is a plurality of nucleic acids, proteins, or small molecules. In some embodiments, the methods further include monitoring transfection efficiency or cell mortality. In some such embodiments, the methods further include adjusting the electric field, the flow rate, a concentration of the at least one cell, a concentration of the at least one molecule, or the chemical composition of the fluid based on the transfection efficiency or cell mortality. In some embodiments of these methods, the fluid channel has a second dimension that is at least 10-fold and at most 1000-fold greater (e.g., 20-, 30-, 40-, 50-, 60-, 70-, 80-, 90-, 100-, 150-, 200-, 250-, 300-, 350-, 400-, 450-, 500-, 550-, 600-, 650-, 700-, 750-, 800-, 850-, 900-, 950-fold greater) than said at least one dimension.

These and other aspects of the present disclosure will become apparent upon a review of the following detailed description and the claims.

### BRIEF DESCRIPTION OF FIGURES

FIG. 1 shows a cross sectional schematic view of a portion of a fluid channel device (i.e., an electroporation device) in accord with an embodiment of the present disclosure.

FIG. 2 shows a cross sectional schematic view of a fluid channel device (i.e., an electroporation device) including a fluid channel system, multiplicity of fluid inputs, output, and a pair of electrodes.

FIG. 3 shows an embodiment of an electroporation device constructed with three layers.

FIG. 4 shows a schematic of a system for control of fluid flow and electrical voltage, and for optically and electrically monitoring controlled electroporation.

FIG. 5 shows a cross-sectional schematic view of an embodiment of an electroporation device.

FIG. 6 shows an exemplary sinusoidal voltage waveform with an amplitude of 10 V and a frequency of 10 kHz.

FIG. 7 shows an exemplary periodic voltage waveform with a high frequency component for permeabilizing cells and a low frequency component for electrophoretically driving charged molecules.

FIG. 8 shows a schematic of a multichannel device made from laminated molded parts.

FIG. 9 shows a perspective view of an embodiment of a fluid interface.

FIG. 10 shows a perspective view of an embodiment of a fluid interface.

FIG. 11 shows a schematic of cell processing function that is integrated with a planar fluidic electroporation device (which, for example, can be a microfluidic chip or alike). The fluidic flow device schematic shows separate regions for magnetic cell selection and electroporation in a planar format.

FIG. 12 shows a system of an independent microfluidic device and a planar electroporation device (e.g., an electroporation chip). In this figure, and in the rest of the disclosure, unless described otherwise in a particular context, the word “chip” is used interchangeably with the word “device” for facilitating the discussion of various aspects.

FIG. 13 shows an automated cell manufacturing platform that incorporates an electroporation system.

FIG. 14 shows a multi-flow device with dynamic control of processes to obtain optimum cell modification parameters.

FIG. 15 shows a schematic of independently controlling and varying the chemical composition of three fluid streams for combinatorial processing.

### DETAILED DESCRIPTION

The present disclosure relates to a system, method, and device for the introduction of a biologically active molecule into a living cell by electroporation. The disclosure allows for monitoring and controlling cell location, motion, and exposure to electric fields between electrode pairs within the device such that each cell is exposed to similar electrical and chemical conditions during electroporation. In an embodiment, an electroporation device contains a fluid channel flanked by two electrodes on opposite sides of the channel to which electrical potentials can be applied to create an electric field across the channel between the electrode pair. In some embodiments, the dimensions of the fluid channel combined with the characteristics of the fluid flow provide sufficient control to maintain individual living cells within the fluid flow at similar positions with respect to proximity to the electrode pair they are passing through. As the living cells flow through the channel between the electrodes, the distance from the cell to each electrode is held to be nearly constant and in a manner that prevents one living cell from shielding another living cell from the applied electric field. Typically, the cell flow is one layer thick in the channel dimension between the opposing electrode pairs so that the cells are independently exposed to the same electrical current formed in the channel when passing between the electrode pairs. The channel, in some embodiments, has no restriction on distance in the other two dimensions of channel length and opposing channel walls not flanked by the electrodes. The cells flow through the channel at a set flux, and these features enable a user to apply precise electric fields to the cell. The strength of the electric field is strong enough to form pores within the membrane of the living cell through which biologically molecules can traverse the cell membrane, but weak enough to not lyse the cell.

The device includes one or more fluid inputs and at least one fluid output. When the device includes a single fluid input, a single laminar fluid stream is created. The single fluid stream contains living cells in combination with biologically active molecules for introduction of the biologically active molecule into the living cell by electroporation. Suitable spacing between the electrodes includes about 2 to 5 times larger than the diameter of the cell, or smaller than approximately two times the typical cell diameter, forcing the cells to pass through the space between the electrodes in a single layer. The living cells in the single fluid flow are maintained in a similar position as other living cells as they pass through the electric field between the electrode pairs, so each cell is exposed to similar electrical and chemical conditions during electroporation. Suitable distance between the electrodes of an electrode pair includes a range of from about 50 micrometers to about 100 micrometers, or less than about 100 micrometers (e.g., 20, 25, 30, 35, 40, 45, 50, 55, 60, 65, 70, 75, 80, 85, 90, 95 micrometers).

When the device includes at least two fluid inputs, multiple laminar sheath fluid streams are created. Each fluid input can accept a separate fluid stream. For example, one stream contains living cells, and another contains the biologically active molecules. Thus, the living cells and the biologically active molecules flow through separate fluid inputs into the channel. The streams are separated by laminar sheath flow. The dimensions of the fluid channel are constructed to accommodate the laminar flow separated streams so that living cells contained in the fluid flow are maintained in a similar position as other living cells as they pass through the electric field between the electrode pairs. In a system with multiple sheath flows, the sheath flows separate the cells from the electrode and channel walls with a constant spacing controlled by the flow rates. The multiple sheath flow devices allow the chemical composition of the fluid on opposite sides of the cell to differ permitting an efficient electrical drive of charged molecules such as DNA and RNA into the cells. The flow of liquid through the channels can be unvarying in time, which simplifies the process and assures that all cells experience the same combination of conditions during electroporation as they pass through the flow channel. Suitable distance between the boundaries of the sheath flow containing the living cells between paired electrodes includes from about 50 micrometers to about 100 micrometers; less than about 100 micrometers (e.g., 20, 25, 30, 35, 40, 45, 50, 55, 60, 65, 70, 75, 80, 85, 90, 95 micrometers); about 2 to 5 times larger than the diameter of the cell; or smaller than approximately two times the typical cell diameter, forcing the cells to pass through the space between the electrodes in a single layer. The device can contain one or more fluid outputs. In this embodiment, the distance between the electrode pairs can be greater than that noted above since the living cells in the sheath stream are maintained in a similar position as other living cells as they pass through the electric field between the electrode pairs by the adjacent sheath flows. A suitable distance between the electrodes of an electrode pair includes from about 50 micrometers to about 500 micrometers. Another advantage of an embodiment of the disclosure is that the user can manipulate the chemical and electrical properties of the environment at different positions along the length of the channel. Furthermore, an embodiment of the disclosure allows the user to monitor various properties of the cells and/or the solution to modify and optimize the flow and voltage parameters in real time.

Channel

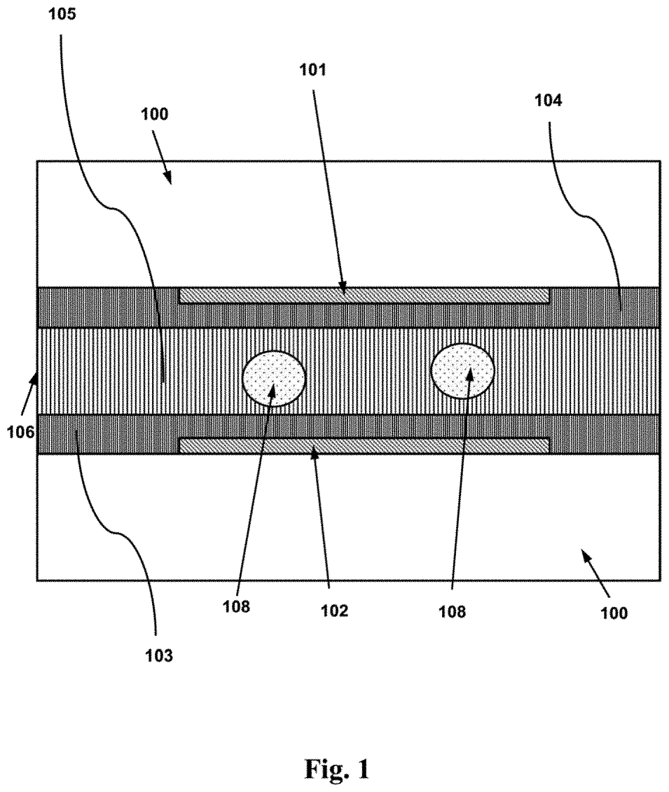

FIG. 1 shows a cross-sectional schematic view of an embodiment of the device. A flow channel 106 lies between two support blocks 100. A positive electrode 101 lies on an inner flow channel surface of upper support block 100 opposite a negative electrode 102 which lies on an inner flow channel surface of lower support block 100. A liquid stream 106 of buffer containing cells 108 and nucleic acids or other biological molecules 110 to be electroporated flows between lower support block 100 and upper support block 100.

By limiting the gap dimension between electrodes to be about 2 to 5 times larger than the diameter of the cell or less than approximately two times the cell diameter, there is not enough physical space for more than one cell in the flowing stream to be located in the channel gap between the electrodes. This controlled gap spacing as well as operating in the laminar flow regime (e.g., no turbulent flow) allows for controlled positioning of a single given cell between the electrodes in this one plane. While the flow channel is narrow in proximity to the electrodes, the channel can be made as wide as necessary in the orthogonal dimension to achieve the desired flow rate of the cells through the channel. Similarly, the length of the channel, in some embodiments, has no restrictions. Control of the distance between the electrodes allows each cell to be isolated or held in a similar position relative to the electrodes. Thus, each cell is essentially subjected to a similar electrical and chemical environment while, at the same time, high total cell throughput is possible. In one embodiment of the device, the channels can be manufactured so that distance between the support blocks 100, and thus the electrodes 101 and 102, can be adjusted to accommodate different types and sizes of living cells. The support blocks 100 on which the electrodes 101 and 102 are mounted can be made of any nonconductive or electrically insulating material, such as glass, plastic, or optically transparent material.

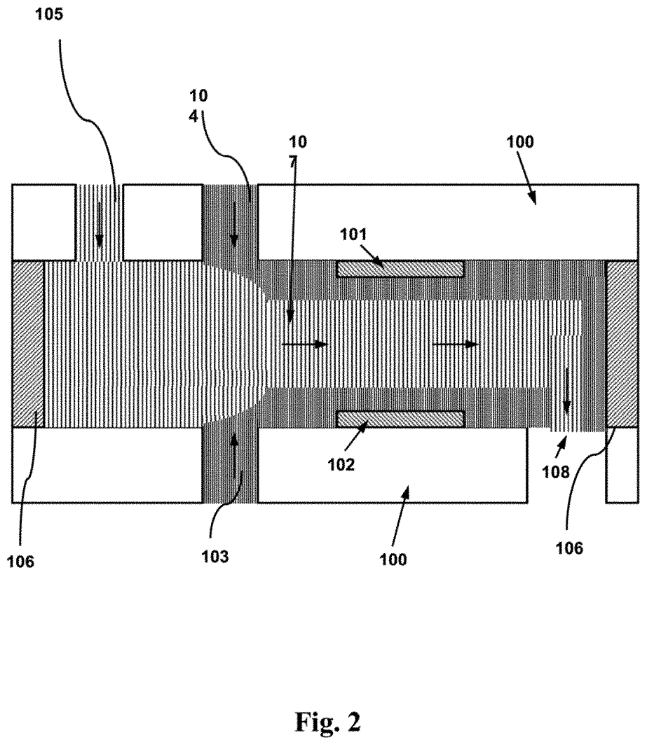

FIG. 2 shows a cross-sectional schematic view of an embodiment of the device. A flow channel 107 lies between support blocks 100 with patterned electrodes; a positive electrode 101 lies opposite a negative electrode 102. A lower liquid stream 103 of a high conductivity buffer containing nucleic acids or other biological molecules to be electroporated flows adjacent the negative electrode 102. An upper liquid stream 104 of a high conductivity buffer flows adjacent the positive electrode 101. A middle liquid stream 105 of a low conductivity buffer containing cells 108 to be electroporated flows between lower liquid stream 103 and upper liquid stream 104. The upper liquid stream 104, middle liquid stream 105, and lower liquid stream 102 are separated by laminar flow.

In FIG. 2, fluid from the inputs 103, 104, and 105 flow through the channel 107 and exit the device via an output 108 flow. Spacers 106 are used to change direction of the flow.

The flow channel can be made in various geometries and can have either a constant or variable width.

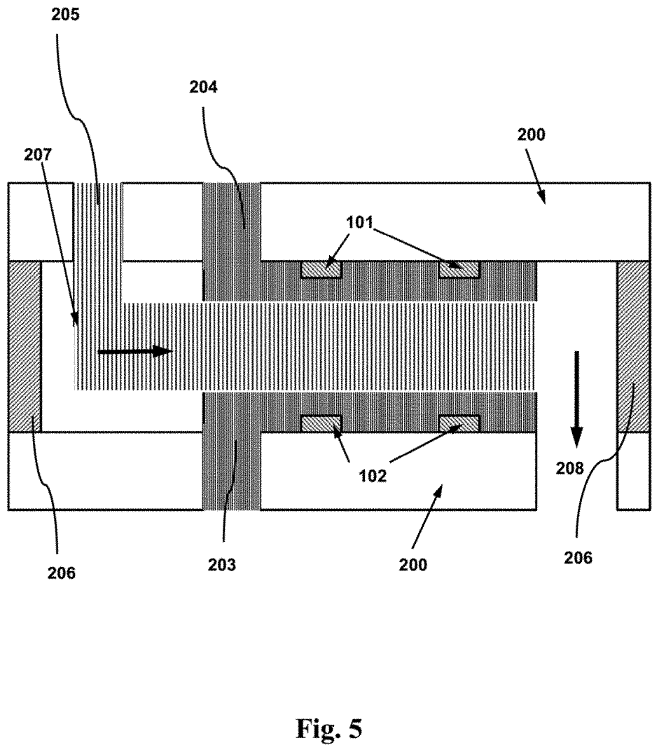

FIG. 5 shows a cross-sectional schematic view of an embodiment of the device. The flow channel 207 lies between support blocks 200 with patterned paired electrodes; two positive electrodes 101 lie opposite two negative electrodes 102. The distance between the two electrodes is selected to be slightly larger than the diameter of the living cells flowing in fluid input 205. While the flow channel is narrow in proximity to the electrodes, the channel can be made as wide as necessary in the orthogonal dimension to achieve the desired flow rate of the cells through the channel. Similarly, the length of the channel has no restrictions in this embodiment. Control of the distance between the paired electrodes allows each cell to be isolated or held in a similar position relative to the paired electrodes, considering the laminar flow established by fluid inputs 203, 204, and 205. Thus, each cell is essentially subjected to a similar electrical and chemical environment while, at the same time, high total cell throughput is possible. Fluid from the inputs 203, 204, and 205 flow through the channel 207 and exits the device via an outlet flow 208. Spacers 206 are used to change direction of the flow.

### Fluid Inputs and Streams

The fluid can flow through the channel at a rate of 0.1 cm/s, with a relevant range of flow rate between 0.001 cm/s and 10 cm/s. The volume of fluid flowing through the channel relates to the cross-sectional area of the flow channel. For example, for a channel 2 cm wide and 100 micrometers high, the volumetric flow rates would be in the range of from about 0.2 microliters/s to 2 milliliters/s.

The device permits the use of multiple inputs of fluid through slits in the channel device to provide flow with different layers of solution composition. The flow rate of two or more fluid streams into the channel can be controlled to create a sheath flow. In one embodiment of the device, the channel 107 (FIG. 2) delivers a low conductivity buffer containing living cells to be electroporated through a liquid sheath. An optional channel 104, on the same side of the device as channel 105, delivers a high conductivity buffer. Another channel 103, located on the opposite side of the device, also delivers high conductivity buffer. In FIG. 2 this buffer contains the biologically active molecules that are to be inserted into the living cells. Various streams of cells or molecules enter the channel via these inputs, and these streams can have the same or different flow rates. If desired, streams with different flow rates adopt laminar flow through the channel. Thus, the streams flow in parallel through the channel and remain largely separated, mixing slowly only through diffusion. In this manner, individual cells in the stream of living cells can be isolated between the electrode pair by the laminar flow of the adjacent fluid streams.

The use of multiple inputs of fluid can prevent various types of fouling or contamination. For example, the molecules or nucleic acids to be inserted into the cells can exist in a separate solution from the cells. This can be useful because certain molecules, like RNA, may not be stable in the vicinity of living cells due to enzymes on the cell surface or cell culture media. Also, it is known that degradation of the electrodes can result in the release of contaminants that are toxic to cells. The separate fluid layers ensure that the cells remain free from contaminants from the electrodes. Further, the cells themselves are kept out of contact with both the surface of the support block and the electrodes preventing possible contamination.

In some embodiments, using separate fluid streams allows maintaining different components in media optimal to them for a longer time. For example, one fluid stream can contain the cells to be electroporated, and instead of keeping the cells in a medium that is best for electroporation efficiency, the cells can be kept in a medium that is optimal for them (e.g., for their survival) before electroporation, and then allowed to mix with an electroporation medium during the actual electroporation time window. After the electroporation is complete, the cells can be switched back into the medium that is optimal for them. This allows minimizing the time the cells are in a medium that is not the best for their well-being. The embodiments disclosed herein thus allow dynamically controlling the chemical environment of the cells and the reagents to be electroporated into the cells separately, for example as a function of time and/or position within the fluid channel.

Alternatively, an embodiment of the device can contain a single fluid input through which a homogenous solution of cells and biologically active molecules enter the channel. The stream consists of a conductive buffer solution containing the biologically active molecules that are to be inserted into the living cells. The biologically active molecules are chosen, in some embodiments, from among the categories of nucleic acids, drug molecules, and other biologically active molecules. Compared to the device having multiple inputs, this might be advantageous in that there is a greater opportunity for the cells and the biologically active molecules to come into contact with one another and could increase the efficiency of transformation.

In one embodiment, the inputs 104, 105 introduce fluid streams to the channel so that the streams turn at an angle before flowing in between the electrodes. In FIG. 2, this angle is shown as 90°, but the angle can be any angle including 0°. In this case, spacer 106 helps to direct the flow from the inlets 104, 105.

Similarly, in one embodiment of the disclosure, the flow turns a corner before exiting the device through the output 108. In FIG. 2, this angle is 90°, but this angle can be any angle, including 0°. In this case, spacer 106 helps to direct the flow to the outlet.

Fluid streams interface to the device via tubing, fittings, interconnects, a manifold, or discreet fluid path connections. One or more of these parts can be part of the fluid interface. The fluid interface serves to reformat the tubing or conduits into the receiving slit-port of the device (e.g., 103, 104, or 105 in FIG. 2). The fluid interface may have changes in surface area as well as varying geometries for delivering fluid to the device. The fluid interface may have features to enhance mixing or maintain laminar flow characteristics. This includes geometric changes that may aid in turbulent flow, diffusion rate changes, or residence time in the flow path. The fluid path may have geometries tailored to avoid the trapping gas (bubbles) or seeding to avoid gas bubble formation due to gas coming out of solution.

The fluid path components may be machined, molded (e.g., injection molding), casted, extruded, or the like. The fluid interface may be fabricated as part of the channel device (one-piece) or bonded (integrated) to the device via a permanent or non-permanent bond.

Alternatively, the fluidic interface could be manufactured as part of the device as one integrated component, for example via injection molding where the device and fluid interface are both formed during the molding process. Sealing between the fluid interface and the device may be hermetic, compression-based, O-ring-based, gasket-based, adhesion-based, fused, luer locked (quick connect), flat bottom compression-based, tapered ferrule-based, frusto-conical compression-based, friction fit, barbed connection, or the like.

Fluid transfer lines may be soft, semi-hard, or hard where the leak tight seal between components are made with connections known to those in the art.

Tubing and fluid conduits may be manufactured via extrusion or molding.

For some manifold designs, portions of the system may not contain tubing and fluid will be routed via the manifold structure.

In one embodiment, the fluid interface to the device is via a leak-tight seal to the planer device with a compressive material such as an O-ring or gasket.

The device can be interfaced to a fluid delivery system. A fluid delivery apparatus or pump is configured to displace fluid from a vessel to establish a fluid flow within the fluid path. The fluid vessel may contain a pure fluid or a solution. The fluid may contain cells, small molecules, or large molecules including chemical entities for the transfection process. The fluid displacement apparatus can provide positive and/or negative displacement of the fluid. This allows fluid to be pushed or pulled through the device and the fluid path components.

The delivery pump includes mechanisms may include peristaltic, syringe, gear pump, diagram, gas pressure (positive or negative), centrifugal, piston, check-valve, or mechanical displacement, hydrostatic or gravity driven flow.

Preferably, the fluid is indirectly displaced by the pump without the liquid directly contacting any of the moving parts of the apparatus, such as, for example, a peristaltic pump acting upon a fluid filled tube. Alternatively, a positive pressure displacement mechanism may be used where a head pressure displaces liquid from a pressurized vessel, or a negative displacement where a vacuum is used to pull liquid into the electroporation device; vacuum via pressure regulator or a peristaltic pump. The use of negative displacement allows for limited system components to be implemented on the inlet side of the device.

When pulling liquid through the device using negative displacement, an intermediate vessel could be used to capture the cells and fluid exiting the device to avoid contact with the pump's negative displacement equipment (for example syringe, peristatic pump tube, flow sensor, or alike).

Conversely, fluid may be directly displaced by an apparatus, when the fluid is displaced by directly contacting any of the moving parts of the apparatus, such as, for example, the plunger of a syringe pump. Alternatively, the syringe pump could pull liquid through the device with the target fluids not traveling to the point of reaching the syringe barrel. The syringe may be reusable or disposable. The syringe may be integrated in the fluid path or connected at the time of use.

Fluid control may be open-loop or may have closed-loop feedback control.

Pumping systems established to date have several weaknesses for controlling flow rate accuracy and precision, and may have performance limitations around controlling stable non-pulsing flows. Controlling of fluid pulsing for the electroporation device is most preferably controlled on the time frame of less than 30 seconds, more preferably less than 10 seconds, and most preferable faster than 1 second. Pulsing control is better than that of 20% for the given time period mentioned in the latter.

For the electroporation device, a preferred embodiment includes the peristaltic pump mechanism and or a gas pressure pump-based mechanism. Both types may operate to pull or push liquid.

Traditional peristaltic pumps suffer from high pulsing delivery because the fix rate of mechanical contact on the pump tube via rollers (or linear compression mechanisms) which continuously alter the cross-section area by compressing the tube resulting in tube ID change. Pulsing results from the cross-sectional change of the tube ID. Additionally, peristaltic pumps suffer from accuracy issues that result from tubing compliance changes and tube wear characteristic changes over time and use. This wear cannot be compensated or adjusted for without direct measurement of the fluid flow rate or measuring the output with a balance or volumetric measurement. Measuring liquid flow rate with a balance is not practical as the then an additional instrument must be added that requires an adequate environment (e.g., temperature, humidity, vibration, and space). Also, the fluid path then becomes dependent on access to the relatively large footprint/space requirement of a balance.

Pressure pumps deliver relatively non-pulsatile flow but suffer from accuracy issues because of fluid path dimensional tolerances, viscosity and temperature changes (fluid and ambient temperature), and liquid height changes as vessels are empty and filled. Measuring liquid flow rate with a balance is not practical as then an additional instrument must be added that requires an adequate environment (e.g., temperature, humidity, vibration, and space). Also, the fluid path then becomes dependent on access to the relatively large footprint/space requirement of a balance.

To counter these limitations a flow-sensor may be implemented to provide closed-loop feedback to the liquid displacement mechanism. Here is proposed the addition of a fluid flow rate sensor (in line with system components) to measure the flow rate in near-real-time with the ability to feedback to the fluid displacement mechanism. For example, a flow rate sensor with a peristaltic pump or a gas pressure control system acting on a fluid vessel. The flow rate sensor may control the fluid displacement continuously or intermittently. The sensor may also be used to measure the flow rate as a check in the case of open-loop operation.

Most preferably, in some embodiments, the sensor does not contact the fluid and is not in communication with the device, tubing, or conduit.

The sensor may be reusable where it is used in conjunction with a disposable fluid component(s). Or the sensor maybe disposable.

Most preferably, the two types of sensor that may be used include, but are not limited to: (1) ultrasonic-based sensor that is in communication with the fluid path (non-contact), which sensor is in communication with a component the liquid is traveling through; and (2) thermal flow sensor that is in communication with the fluid path (non-contact), which sensor is in communication with a component the liquid is traveling through.

The sensors may be re-used where they temporarily interfaced with a fluid path component that is to be changed, or the sensor may be part of the path and be disposable in nature. In some embodiments, the disposable sensor is integrated in the fluid path.

Interfacing of the liquid entering the device may occur via one or more components, such as a tube or conduit, and/or a fluid interface. The fluid component may comprise one or more features that allow for distributing or altering the flow profile and path of the fluid. This component may a wetted path where the cross-section area and shape may be varying from that of the fluid component exiting cross sectional area or shape. The fluid path change may be part of an assembly or may be molded as part of the electroporation device.

This may include geometric shape(s) that may be redistribute or format the liquid flow from the tube conduit to a format that is compatible with the device inlet. This architecture of the fluid path depends on the incoming fluid source tubing, fitting, or fluid interface as well as the device fluid inlet shape.

The fluid interface component may be composed of one or more fluid paths and is not limited to the location or number of inlet or outlet features.

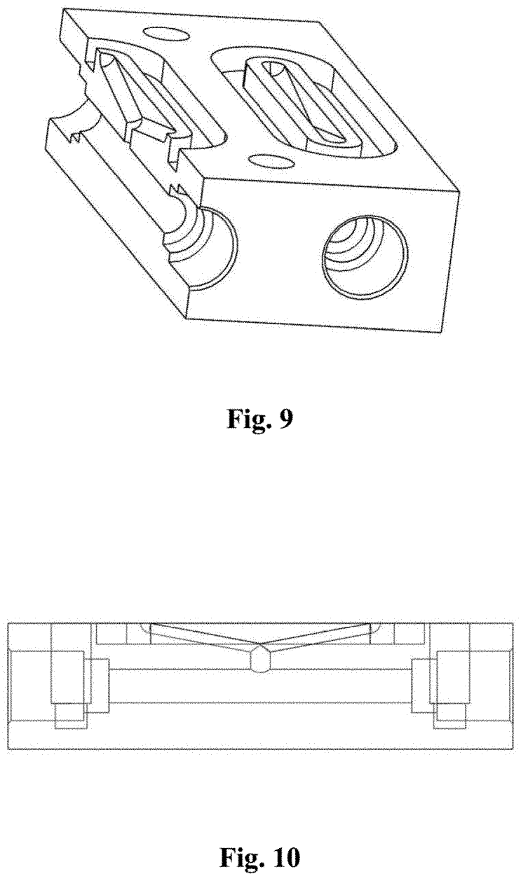

Schematic depictions of two embodiments of the fluid interface are provided in FIG. 9 and FIG. 10. A fluidic interface may serve to allow for various formats and types fluid components to make a fluid seal to the microfluidic device inlet. The device inlet may be a circular shape or may have a non-cylindrical geometry or shape. A fluidic interface component may for example allow for one or more incoming fluid lines or conduits to connect to the fluidic interface inlet where the fluid may then traverse a changed cross-section or geometric shape, followed by the fluid exiting the fluidic interface in a cross-section or shape that matches the device fluid inlet geometry. The fluid device inlet geometry would correspond to the fluid interface component output geometry. For example, the fluidic interface may serve to allow for a traditional tube to then supply fluid to a split on the device. The depictions in the figures are only exemplary, as interfacing the fluid can be accomplished in many ways (e.g., different geometric shapes for different types of conduits/tubes).

Cells can be manipulated post electroporation. In some embodiments, after electroporation, cells are transferred from the PA to a sterile, multi-well dish or T-flask and allowed to recover for 30-40 minutes at 37° C. The cells are suspended in standard cell medium and either cultured for immediate use or cryopreserved.

In some embodiments, the electroporation device is interfaced to a receiving station that serves the purpose of making one or more connections providing the means to make a leak tight fluidic connection. Such an interface station may also service to make electrical connections.

The receiving station may (1) include ability to make one or more fluidic leak tight connections; (2) include ability to make one or more electrical connections; (3) contain regions allowing for optics or a path for allowing external optics access to the device; (4) have all wetted components that are disposable in nature and compatible with a means of sterilization; (5) have fluid that is isolated via various fluid regions inline valves or more preferably via a non-contact mechanism such as a pinch valve, or the pump may include a way to isolate flow, for example when use of a peristaltic mechanism is utilized (e.g., he wheel may be positioned to pinch a tube or pump tube closed); (6) have conduits, tubing and fluid components that link via barbed or compression fittings; and/or (7) encompass using welding and part melting for manufacturing fluid assemblies.

Cells and the bioactive materials may be presented to the device via several approaches. They may be injected via a robotic fluid handling platform or injection system or connected via biocompatible containers. Bioprocess containers include polymer bags, T-flasks, conical tubes, media bottles, well plates, or the like. These vessels may be one time use or reusable when proper sterilization is performed. Connections to the fluid delivery path may be achieved by compression seals, threaded connections, clamping compression, luer lock mechanisms, O-ring seals, friction seals, gaskets seals, clamping, or similar connections. In the case of pneumatic displacement, the container itself may be pressurized or be contained inside a pressurized vessel.

In an embodiment, the cells may be presented to the device by custom cartridges that interface to the pumping or fluid manipulation system.

### Fluid Output

An exemplary fluid outlet 108 is shown in FIG. 2. After electroporation, a mixture of all the fluid streams can leave the device via this outlet. The solution may be transferred to sterile polymer bags, T-flasks, conical tubes, media bottles, well plates, or the like and allowed to recover at 37° C. The cells may then be re-suspended in standard tissue culture medium and plated for immediate use in cellular assays, cryopreserved for future use, or used as desired.

Electrodes with Temporal and Spatial Control of Electric Fields

The separation between electrodes located across the thickness of the fluidic device is small, therefore requiring an applied voltage of only a few volts to perform the electroporation. This contrasts with the need for voltages up to several thousand volts that are normally required for standard electroporation. For example, it is known in the literature that a transmembrane electric field of less than 1 kV/cm is required to porate the cell membrane (Weaver and Chizmadzhev, 1996). However, for a distance between the electrode pairs of 100 micrometers, this requires approximately a 5 V potential difference to porate an average mammalian cell in accord with the present device. Suitable voltage differences across a living mammalian cell include the following range: 0.1 V to 10 V. For example, for a distance between the electrodes of 100 micrometers this range corresponds to an electric field of 10 V/cm to 1000 V/cm.

The flow channel can have one or several electrically independent electrode pairs. For example, it can have four sets of electrode pairs 101. Connections to the electrodes are made by using clips or conduction adhesive to connect these to a variable-voltage power supply, function generator, computer via a data acquisition card or amplifier, or batteries with a voltage divider. An ammeter can be used to monitor the current flowing between any pair of electrodes for monitoring and controlling the process.

The electrodes can be configured to apply either a constant, pulsating, or continuously time varying voltage perpendicular to the direction of flow or along the direction of flow. If a pulsating voltage is desired, a pulse duration from about 0.01 millisecond to about 100 milliseconds is suitable. The plurality of electrode pairs can be patterned to create spatially and temporally varying electric fields. The electrodes may be patterned using a photomask in the photolithographic process or by a shadow mask in the sputtering or deposition process. Patterning allows for the fabrication of electrodes with varying geometric shape. The variation of the shape combined with the fluid flow characteristics provides for controlling the time that cells are subject to the electric field.

The invention provides for the ability to pattern electrodes at different locations on the surface of the flow channel that can be individually connected to various electrical sources, where the electrical sources can have different voltage and current characteristics. The disclosed planar fluid systems consisting of electrically insulating material(s) enable the patterning of various electrode structures.

In various embodiments, any one or more of the following can be done: (1) one electrode or group of electrodes can be activated with time-dependent voltage characteristics to open pores in the cells; (2) another electrode or group of electrodes can be activated to drive charged molecules into cells; (3) another electrode or group of electrodes can be used to measure the electrical properties of the cell-containing fluid; (4) another electrode or group of electrodes can be used to concentrate nucleic acids or other molecules at the interface between fluid layers of varying conductivity; (5) another electrode or group of electrodes can be activated to move the cells actively, or passively by a creating flow in the fluid, to a prescribed location in the flow channel for cell sorting or other purposes; and (6) another electrode or group of electrodes can be activated to rotate the cells to increase the surface area exposed for electroporation.

Importantly, some of the disclosed embodiments permit the application of an arbitrary time-varying voltage to different electrodes. The voltage signals can be formed by computer generation of the desired time varying waveform, which is converted to an applied voltage by digital to analog conversion and amplification to the desired voltage range.

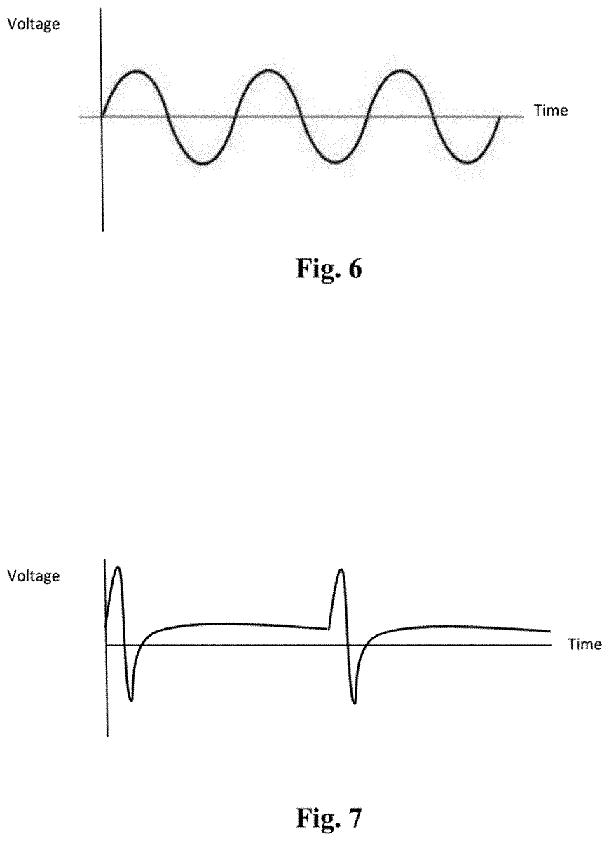

A simple waveform would be a sinusoidal voltage of prescribed frequency as shown in FIG. 6. The amplitude of the waveform needs to be sufficient to permeabilize the cells. This, in some embodiments, requires a voltage drop of approximately 1 V over the typical 10 micrometers size of a mammalian cell within the fluidic device. This implies that the amplitude of the voltage waveform could be about 5V, with a range extending from 0.1 V to 100 V depending on the depth of the fluidic device (e.g., chip) and the ionic composition of the fluid layers. The frequency of the pulse depends on the impedance characteristics of the circuit, specifically on the capacitive aspects of the so-called double layer that is known to form at the surface of the electrode due to the presence of free moving ions in the aqueous solution as well as the resistance of the fluid, or fluid layers of varying conductivity. The impedance of the capacitive double layer depends inversely on the frequency. Consequently, the frequency should preferably be around 10 kHz so that the impedance of the fluid layers dominates, leading to most of the voltage change occurring within the fluid layer and not at the electrode-electrolyte interface. The frequency might range from 100 Hz to 1 MHz depending on the fluidic device dimensions and the ionic composition of the fluid layers. The impedance of the circuit may depend on a complicated manner on the ionic conductivity of the fluid layers. The resistance of the fluid scales inversely with the ionic concentration, while the double layer capacitance is proportional to the ionic concentration raised to some power. The circuit at the electrolyte-electrode interface is often approximated as a capacitor due to the double layer in parallel with a frequency dependent impedance that is in series with a resistance due to charge transferred across the electrode (referred to as the Randles equivalent circuit model). The ability to control the time variation of the voltage means that the current charging the double layer and the current due to charge transferred across the electrodes may be modulated according to the optimum configuration for electroporating the cells. The voltage waveform could also be composed of the sum of a sinusoidal wave in addition to a constant DC voltage offset, resulting in a net flow of current.

Another periodic waveform, according to some embodiments, has a short duration voltage to open pores followed by a lower voltage of longer duration to move charged molecules into proximity to the cells as shown in FIG. 7. The movement of the charged molecule can be due to an electrophoretic force, or due to electrophoresis from a net fluid motion induced by the electrodes, or due to a dielectrophoretic force on the charged molecule or cell.

The continuous repeating nature of the waveform is useful for the continuous flow systems. The applied voltage can vary from positive to negative or remain at zero or another constant voltage for portions of the waveform.

A waveform of arbitrary shape can be created by adding together any number of sinusoidal waveforms each with their own frequency and amplitude, in addition to a constant voltage offset.

The net time-average voltage can be chosen to be positive, negative, or zero providing the ability to control the net direction of charge flow. This would be of utility for controlling surface electrochemistry on electrodes and for directing charged molecules in a chosen direction.

The waveform may also be chosen to open pores in the cells or cell nucleus and allow time for diffusion of neutral molecules into the cells before another pore-opening voltage application.

In some embodiments, the spatial arrangement of sets of counterpart electrodes across the surfaces of the fluid channel allows creating an electric field within the fluid channel that varies as a function of time and position without a need for a user to create discrete electrical pulses (e.g., via multiple voltage suppliers providing a waveform to each set of counterpart electrodes, which can be a sinusoidal waveform for any set, and which can be different between the different sets).

Electrodes can be patterned by a variety of methods, including ink jet printing, silk screening, lithographic patterning, vapor deposition through a shadow mask and other methods for patterning electrical conducting material on a variety of substrates including plastics.

### Manufacturing the Device

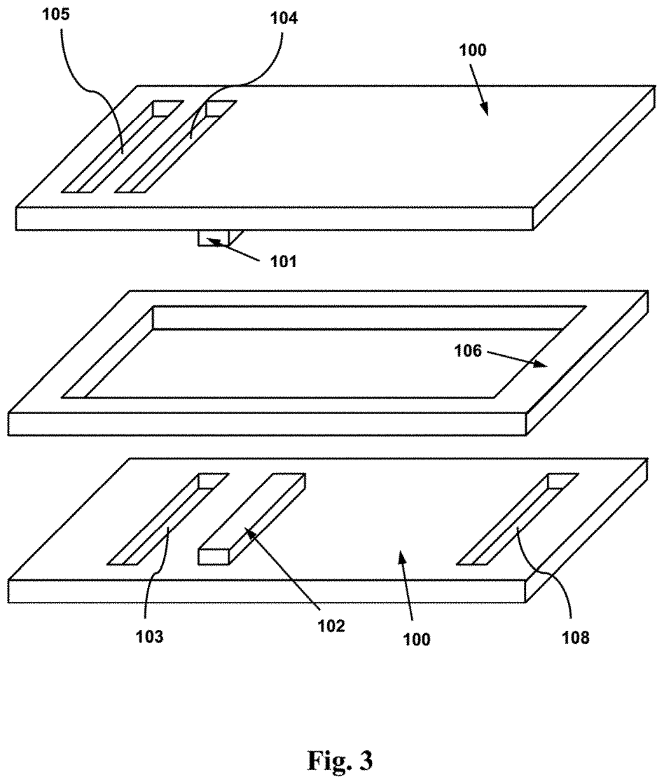

Some embodiments of the device are constructed from a three-layer stack of polymer substrates or plastics as shown in FIG. 3. All three layers are laser cut with a small beam spot, high resolution CO₂ laser. The layers on which the electrodes are fixed are cut from 1 mm thick acrylic slabs, creating opposite surfaces of the channel. A middle layer 106 defines the distance between the electrode pairs 101, 102. In the embodiment shown in FIG. 3, the three dimensions of the layers are the same. Although it is most practical for the layers to be the same in dimensions in the plane that the stream flows, these dimensions can be different from one another. One way to manufacture these layers is to use a laser to cut acrylic pieces to microscope format 25×75 mm, add fluid inlet slits or ports 103, 104, 105 to support blocks 100, respectively and add alignment holes 109 to facilitate assembly. A thin film electrode (50 nm) of a gold-palladium (Au/Pd) mixture is deposited by physical vapor deposition on the inside surface of each acrylic piece. The 100-micron thick middle layer 106 polymer film with medical adhesive on each side is cut to shape and receives the corresponding alignment holes via the laser cutting process. After laser cutting, the three pieces are placed on a jig containing alignment pins corresponding to the alignment holes in each layer. The sandwich assembly is then compression-bonded in a press. This two-step process of laser cutting and compression assembly is amenable to mass production and allows for a cost-effective consumable to be created. The process can be used to manufacture hundreds of thousands of devices per year. This contrasts with many other types of standard non-electroporation microfluidic devices that typically require expensive capital equipment and a large number of chemical processing steps.

Alignment of the device layers may be conducted by optical positioning or a physical means such as datum pads, alignment pins, or structures. The device layers may have receiving features for use with a jig alignment piece or system. Alternatively, the alignment features may reside in the device layers as so no jig or peripheral alignment system is necessary. These may include pin-like structures or features that snap together.

The flow cell could also be produced by an injection molding process, where the volume can scale to millions of single-use devices per year, using one injection molding press with a multi-cavity mold.

This disclosure allows for architectures for manufacturing the device that are readily amenable to injection molding. In this device, all the layers may be formed via injection molding. The fluidic channel may be formed in one layer at full depth or, alternatively, the channel may span two or more layers, where the full depth is achieved upon assembly. Injection ports may be created via core pins. Alternatively, the fluid inlets may be added post molding as a secondary operation or structure. The layers may be molded from the planar surface or from the edges. Appropriate and efficient part release from the mold cavity is known in the art.

The molded layers may be assembled together through mechanical connection, adhesion, bonding, welding (including ultrasonic and laser), fusing, melting, or the like. Additionally, there may be another material between the layers for connection and sealing such as, but not limited to, a gasket, O-ring, washer, or the like. Alternatively, sealing can be achieved through press tight or bonding features.

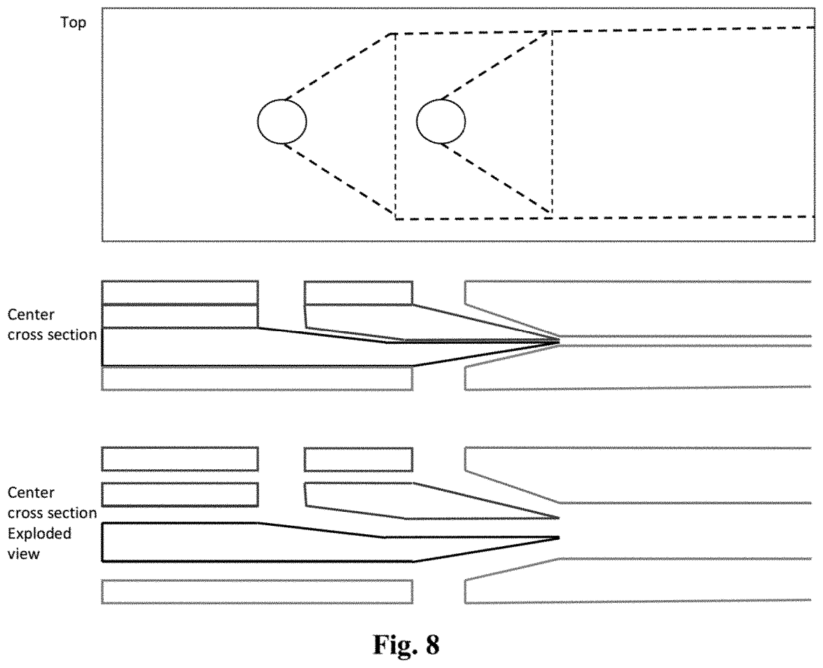

FIG. 8 depicts a three-channel planar laminar flow device that can be formed from four molded plastic components. Not all views are shown in this diagram, but the channels and fluid delivery are incorporated in the structures. Circular entrance ports can be connected with various fittings to conventional tubing such as that from an automated cell manufacturing platform. Low cost manufacturing methods are desirable because the flow cell and material that comes in contact with cell-containing media should typically be discarded after one use to prevent cross-contamination. There are many ways to injection mold including using one mold or more than one over molding technique. Multiple layers may also be bonded post molding using, but not limited to, such techniques as ultrasonic, laser, thermal heat compression, adhesion, or alike.

In another embodiment, the fluid channel may reside in one layer and the opposing sealing structure is a non-injected molded part such as a film, tape, or planar material containing necessary fluid inlets.

In another embodiment, the device may be created by three-dimensional printing or additive manufacturing processes. Other fabrication techniques include compression molding, casting, and embossing.

In another embodiment, devices are made from glass via lithography and wet or dry etching. Alternatively, the devices may be physically machine via computer numeric control (CNC) or ultrasonic machining.

In other embodiments, the devices can be made from various materials, such as, for example, where at least one layer is glass, where at least one layer is plastic, where one of the layers is optically transparent, or where the channel material is electrically insulating.

### Manufacturing the Electrodes

The formation of patterned electrodes on the flow channel surface can be accomplished with a variety of readily available techniques. One method is to use the process of sputtering for deposition of a metallic conducting layer such as gold, platinum, aluminum, palladium, other metals, or alloys of multiple metals. Gold-palladium is an example of a metallic alloy that can be used to compose the electrodes. The electrodes can be made of an optically transparent material to allow observation of the motion of the living cells in the fluid channel of the device. To generate transparent conducting layers, films of indium-tin oxide (ITO) are frequently used. After metal deposition, these conducting layers can be patterned by masking and etching to remove material where it is not wanted to form the desired patterned electrode shapes. Appropriate masks may be formed from photoresist using common photolithographic exposure processes.

Another method for forming electrodes is to deposit electrically conducing films made of metals or other conducting layers such as ITO. By depositing them through a prepositioned mask, sometimes called a shadow mask, the films are positioned in proximity to the surface to be coated so that the conducting layer reaches the surface only where previously opened regions have been formed in the mask. In addition, a related technique called “lift-off” can be used, in which a patterned photoresist layer can be used to shape the pattern of deposited conducing material.

The deposition of layers of conducing ink can be performed by brushing or spraying, followed by heating to form patterned conducting films.

These thin film patterning processes are well known to those skilled in the art. In this case, the thickness of the films is desired to be in the range of from 5 nm to 5 micrometers, with a preferred range of from 10 nm to 100 nm.

In one embodiment of the device, electrodes can be formed by inlaying wires in grooves formed in the support block (e.g., 100 in FIG. 1) instead of affixing the electrodes to the support blocks. In this embodiment, grooves are machined into the support block, for example a plastic support block, and the electrodes are metal. Preferably, the electrodes are gold or a gold-plated metal. The wire is then glued into the groove.

An embodiment of a system includes an electroporation device, fluid delivery system including a pump, temperature control and optical and electrical monitor of the cells to obtain real-time feedback on the cell modification process. Feedback can be obtained by monitoring the electrical current passing between the two electrodes to provide information about living cell modifications, imaging of the living cells to provide information about living cell modifications or monitoring fluorescence of the living cells to provide information about living cell modifications.

An embodiment includes a system for inserting a biologically active molecule into a living cell, which system includes an electroporation device capable of performing a cell modification process including inserting a biologically active molecule into a living cell contained in a fluid flow by flowing fluid including living cells and biologically active molecules through a channel between two electrodes, each electrode disposed on opposite sides of the channel; passing the cells through a space between the two electrodes in a single layer so a living cell in the fluid flow is maintained in a similar position as other living cells in the fluid flow as they pass between the two electrodes; and applying an electric voltage across the two electrodes while the living cell is passing between the two electrodes in a manner that prevents one living cell from shielding another living cell from the applied electric field, in which the strength of the electric field to which the living cell is exposed is sufficient to form pores within the membrane of the living cell through which the biologically active molecule can traverse the cell membrane, but not lyse the living cell; a fluid delivery system including a fluid source and a fluid pump in fluid communication with the electroporation device; an electrical current source in electrical communication with the pair of electrodes; a temperature control in thermal communication with in the fluid flow; and an optical and electrical monitor of the living cell capable of obtaining real-time feedback on the cell modification process.

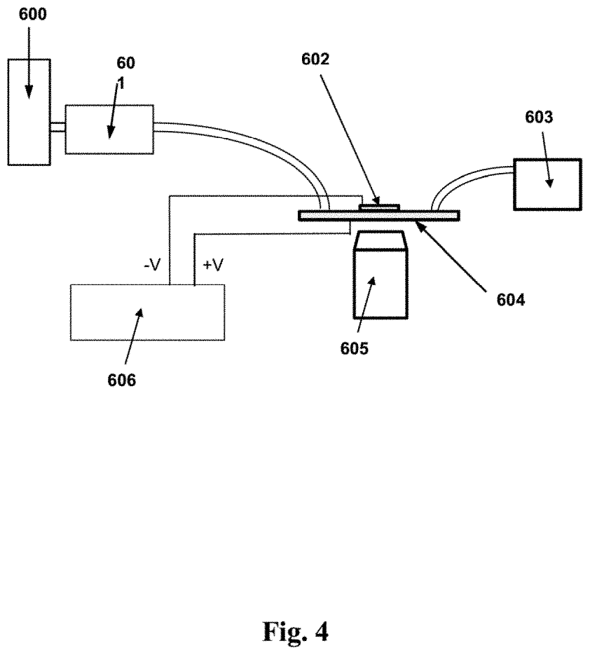

One advantage to the electroporation device over the prior art is the ability to optically and electrically monitor the cells to obtain real-time feedback on the cell modification process. FIG. 4 illustrates one embodiment of the device: a microfluidic electroporation system with an observation microscope 605. Accordingly, the fluid flow controller 601 or voltage controller 606 can be adjusted as required to optimize the process efficiency and cell viability. In this embodiment, the microscope is positioned so that it views a reservoir 602 that contains biologically active material. For example, this could be nucleic acids. The fluid from input cell reservoir 600 flows through the channel of the microfluidic electroporation device 604 and across the field of view of the microscope 605, and into a cell collection reservoir 603, thus enabling the user make adjustments as necessary to improve the efficiency of transformation.

Temperature control of the solutions or materials in contact with the fluids may be implemented at any instance(s) in the system, including heating and cooling. This may include static control or temperature cycling.

The device can be interfaced to a fluid delivery system. A fluid delivery apparatus or pump operating with flow controller 601 is configured to displace, preferably, indirectly displace, the fluid from the input cell reservoir 600 to establish a fluid flow within the fluid path. The fluid displacement apparatus can provide positive and/or negative displacement of the fluid. The delivery pump includes mechanisms based on peristalsis, pneumatics (pressure displacement), hydraulics, pistons, vacuum, centrifugal force, manual or mechanic pressure from a syringe, and the like. Preferably, the fluid is indirectly displaced by the pump without the fluid directly contacting any of the moving parts of the apparatus, such as, for example, a peristaltic pump acting upon a fluid filled tube. Alternatively, a pneumatic displacement mechanism may be used where a head pressure displaces liquid from a pressurized vessel. Conversely, fluid may be directly displaced by an apparatus, when the fluid is displaced by directly contacting any of the moving parts of the apparatus, such as, for example, the plunger of a syringe pump.

The pump may include a flow sensor for monitoring the flow rate or the flow sensor may provide closed loop feedback to the pump control system. The closed loop feedback can ensure accuracy and reduce pulsing. The pump displaces fluid contained in flexible tubing to create a fluid stream. The system may operate with an inline flow sensor configured to directly measure the fluid flow rate as the fluid passes the sensor. The system, in some embodiments, includes a feedback control in communication with the fluid displacement apparatus and the inline flow sensor. The inline flow sensor measures the flow and communicates with a feedback control mechanism. Suitable types of flow sensor mechanisms include thermal pulse, ultrasonic wave, acoustic wave, mechanical, and the like. The inline sensor may be mechanical-based, electrical-based, motion-based, or microelectromechanical systems (MEMS)-based. The sensor mechanism may be thermal, ultrasonic or acoustic, electromagnetic, or differential pressure. One example of a sensor suitable for use in accord with the present disclosure is a thermal-type flow sensor where the sensor typically has a substrate that includes a heating element and a proximate heat-receiving element or two. When two sensing elements are used, they are preferably positioned at upstream and downstream sides of the heating element relative to the direction of the fluid (liquid or gas) flow to be measured. When fluid flows along the substrate, it is heated by the heating element at the upstream side and the heat is then transferred non-symmetrically to the heat-receiving elements on either side of the heating element. Because the level of non-symmetry depends on the rate of fluid flow and that non-symmetry can be sensed electronically, such a flow sensor can be used to determine the rate and the cumulative amount of the fluid flow. This mechanism allows the flow to be measured in either direction. In one preferred embodiment, the temperature sensors and the heating element are in thermal contact with the exterior of the fluid transporting tube and as the fluid stream only contacts the internal surfaces of the tube, the fluid media avoids direct contact with the sensor and heating elements. This format type allows highly accurate and highly sensitive flow measurements to be performed.

Integration of Fluidic Cell Processing with the Electroporation Device

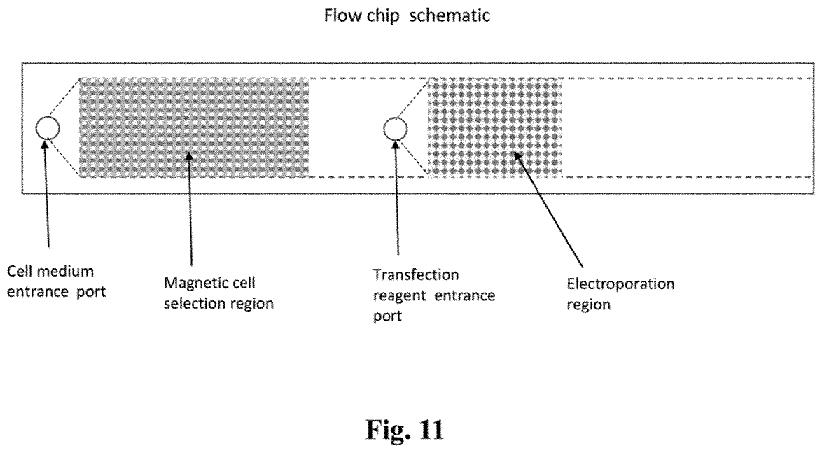

Integration of fluidic cell processing with the electroporation device (e.g., chip) allows building greater function into a system. For example, the multi-channel flow device can incorporate the ability to utilize magnetic bead sorting approaches to select the cells to be processed by electroporation. As an example, FIG. 11 shows a fluidic flow device schematic depicting separate regions for magnetic cell selection and electroporation in a planar format.

Optical transparency of the flow device enables optical monitoring of the processes. Materials may include, but are not limited to, Glass, quartz, polymer, metal films on substrate transparent substrates.

Selection of a variety of T-cells and B-cells can be achieved using magnetic beads conjugated with specific antibodies for the given cell type. There are several commercial manufacturers of superparamagnetic beads including Dynal and Seradyn of a variety of different sizes, typically between 2 and 5 microns. These beads can be used for the positive selection or depletion from flow of CD8+, CD3+, CD4+, and CD19+ cells, for example. The force (F) on a magnetic particle inside a magnetic field depends on the volume (V) of the particle, the difference in magnetic susceptibility between the particle and the surrounding fluid (Δχ), and on the absolute strength and the gradient of the magnetic field (B): F=V·Δχ(B·∇)B. By establishing a large magnetic field and a large magnetic field gradient within the fluidic device, cells that have bound magnetic beads conjugated with the appropriate antibody can be held stationary relative to the flow. In this case, the magnetic force must be stronger than the drag force on the bead from the flow and able to overcome the bead's random diffusive motion.

To establish the sufficient magnetic field within the fluidic device, small neodymium iron boron (NdFeB) magnets featuring magnetic flux densities of up to 500 mT at the pole surface can be placed in proximity to the planar surface. Commercially available versions of these magnets allow for the manipulation of magnetic particles or cells inside a microchannel even when the magnet is placed at several mm distance from the channel. Removing the magnet from proximity of the surface will release the cells. These magnets can be purchased in a variety of sizes ranging from 0.01 to 10 cm in diameter and different geometrical shapes including cylinders, cubes, rings, etc. Also, commercial electromagnets may be used to establish the necessary field and gradient though Joule heating from the relatively high current make these more problematic in small volume applications. It is also possible to fabricate an electrode on the planar surface by the deposition of a conducting metal like gold or platinum. Additionally, the magnetic field within the fluid can be enhanced in the presence of an external magnetic field by depositing and patterning magnetic metals (typically nickel or iron) within the fluid layer. Removing the magnet from proximity of the surface will release the cells. Alternatively, the device and or the magnetic source maybe movable.

Measuring properties of cells upstream and downstream to measure the effectiveness of the electroporation. This requires flow and the ability to constantly monitor the electrical properties (e. g. resistance) of the cell-containing liquid. One could also monitor the change of resistance during the electroporating pulse, where the time-dependent I-V relationship would provide information on the effectiveness of the electroporation process. This is unlike other systems that may exist that do not monitor the effects during the continuous flowing process.

Post Electroporation Cell Manipulation: After electroporation, cells may be moved to an additional region in the device for secondary processing or transferred. The cells may be transferred from the device fluid outlet (or fluid interface component) to a sterile, multi-well dish or vessel and exposed to a secondary set of conditions. For example, to be exposed to for 30-40 minutes at 37° C. The cells are suspended in cell medium and either cultured for immediate use or cryopreserved.

The system may use magnetic bead or microfluidic pillar affinity separation to enrich selected cell types for electroporation and transfection. The input is white blood cells collected from the patient and the output is to a conventional cell culture bag for amplification.

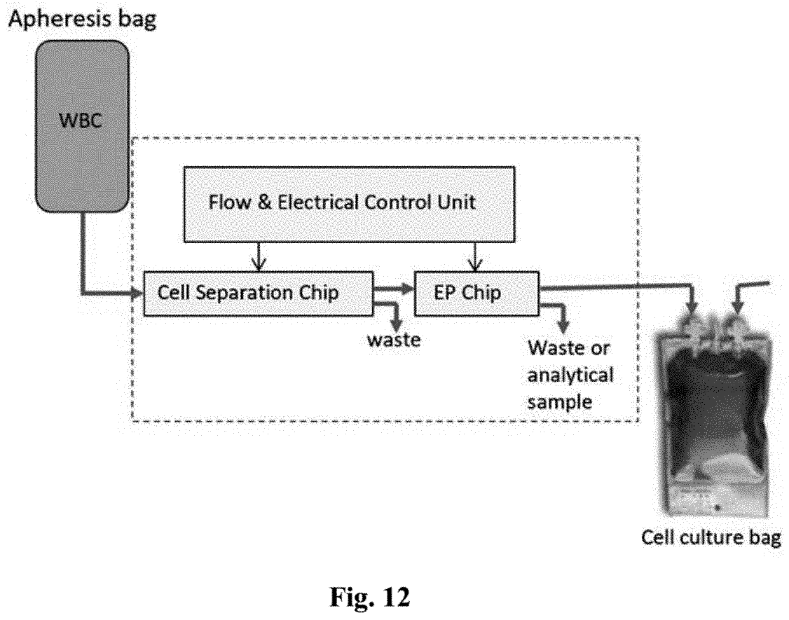

Example process and components are shown in FIG. 12.

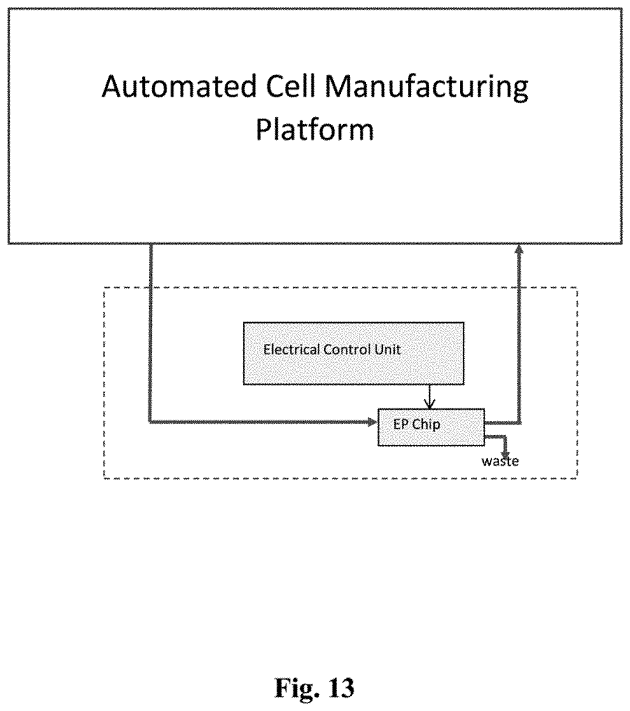

Additionally, the parts or portions of the process may be connected to other processing equipment or stages of a process. For example, in FIG. 13 the electroporation device and components may be implemented with more traditional cell processing hardware.

There is a need for improved methods for selecting the best receptor molecules with which to modify immune system cells for developing treatments for individualized and precisely targeted immunotherapies. Using a flow-based electroporation system it is possible to change the material that one provides to the inputs in time so that one can create many differing combinations of cells and inserted molecules for the purpose of creating libraries of candidate cell treatments for diseases such as cancer. By selection from this library doctors can select the best type of modified cell for treating a specific person's disease. In addition, our multi-input flow-bases electroporation system can be used to create or manufacture specifically designed combinations of cells with differing chemical modifications as prescribed for an individual patient. In this way, the disclosed methods can facilitate a highly individualized immunotherapy-based treatment of many differing types of cancer and disease.

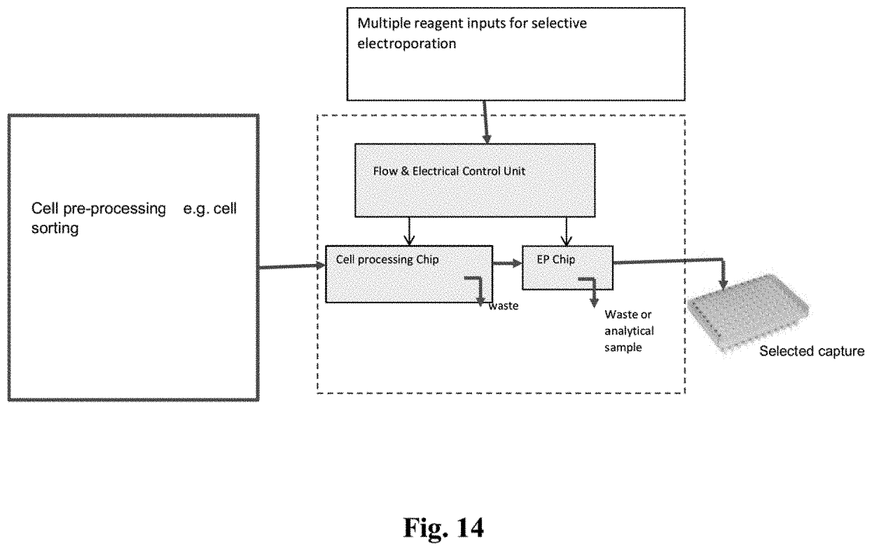

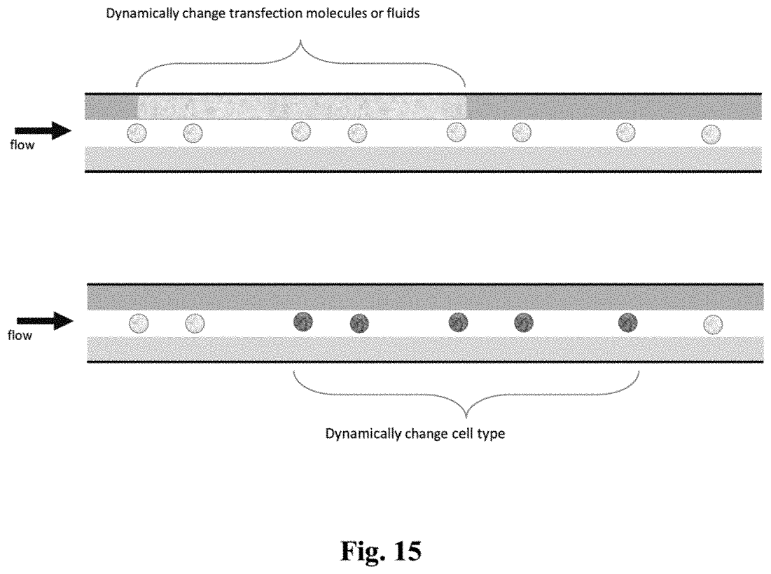

Shown in FIG. 14 is a configuration of a versatile system for rapid controlled processing of cells in the electroporation device. There are numerous advantages that arise from the ability to control and vary the multiple fluid inputs during the electroporation process. By changing the composition of the various fluid streams as a function of time during the electroporation, and collecting the resulting material into different collection vessels, as shown in FIG. 14, it is possible to collect and evaluate a range of electroporation conditions to determine, for example, the best conditions for transfection of a particular cell type or to change the molecules in the stream to evaluate a range of biomolecules to determine which could be valuable for therapeutic use. Such a combinatorial process could greatly reduce the time required to test new compounds and develop cell-based therapies. FIG. 15 indicates that the chemical properties or contents of the fluid streams can be independently varied and controlled to controllably vary the electroporation conditions. Chemical compositions may include, but are not limited to buffers, salts, acids, bases, carbohydrates, peptides, proteins, lipids, and small drug molecules.

Another advantage of this capability is to enable handling small volumes of fluid or small samples of cells and reagents for research. This is particularly valuable in research where the cells may be rare, or the biomolecules may be rare or precious. Such volumes include picoliters, nanoliters, microliters, and milliliter volumes.

Independently controlling the composition of the fluid streams includes possible microfluidic integration of functions for pre and post processing. FIG. 11 shows fluid inputs at different positions along the flow channel. This enables, for example, different chemical conditions to be created before, during, and/or after electroporation. This can be done on the same device (e.g., chip) as show in FIG. 11 or on separate but connected chips as shown in FIG. 14. An important example of this capability includes treatment or selection of cells before electroporation.

Another important application is to maintain different chemical conditions for the cells before, during, or after electroporation. This is valuable, for example, because the conditions for effective electroporation and molecular transfer to the cell may be unhealthy or undesirable for the cells in the longer term. Therefore, changing the chemical composition of the fluid following electroporation enables changing the fluid conditions for the cells immediately after electroporation. This can be done, for example, by flowing in varying chemical-containing solutions such as a nutrient containing medium in which the cells can be maintained effectively for longer times. This nutrient medium can simply be introduced downstream of the electroporation region to dilute the fluid used during the electroporation and establish conditions in which the cells can remain viable or functional for a longer time.

There are numerous other capabilities for enhancing the value of electroporated samples by efficiently processing the material or maintaining different chemical conditions before or after the electroporation process in such integrated systems.

Shown in FIG. 15 is a diagram illustrating two possibilities for the changing of input material in the flow streams to create selected and varying combinations of cells and reagents, to be combined to create modified cells.

### EXAMPLES

The disclosure will be further illustrated with reference to the following specific examples. These examples are given by way of illustration and are not meant to limit the disclosure or the claims that follow.

### Example 1: Electroporation of Mammalian Cells with a DNA Plasmid

This example describes an embodiment where the flow electroporation device is used to electroporate mammalian cells with a DNA plasmid. Chinese hamster ovary (CHO-K1) cells (ATCC) are electroporated with a plasmid that expresses green fluorescent protein using the flow-through electroporation device described. Cell viability can be determined based on the uptake of propidium iodide. The electroporation efficiency can be determined using fluorescent observation of the number of cells that express the green fluorescent protein relative to the total number of cells.

The cells are cultured in an incubator at 37° C. and 5% CO₂. The cells can be cultured in a synthetic medium, such as Dulbecco's modified Eagle's Minimum Essential Medium (DMEM, Sigma, St. Louis, Mo.) supplemented with 10% fetal bovine serum (Sigma) and 100 mg/mL streptomycin (Sigma). When the cell suspension density reaches a certain value, for example, 2×10⁶ cells/mL, the cell suspension is diluted with additional culture medium. Prior to introduction into the device, a 10 mL sample of the suspension is centrifuged at 300 g for 5 min. The supernatant is discarded, and the cells are re-suspended in a low conductivity buffer (described below). The cell suspension density for electroporation is preferably 1×10⁸ cells/mL with a range between 1×10⁷ and 1×10⁹ cells/mL.

The low conductivity buffer is composed of 0.8 mM Na₂HPO_{4,} 0.2 mM KH₂PO₄, 0.1 mM MgSO₄.7H₂O, and 250 mM sucrose, at a pH of 7.4. This buffer is made by dissolving 0.1136 g of Na₂HPO₄, 0.0272 g of KH₂PO₄, 0.02465 g of MgSO₄.7H₂O, and 85.575 g of sucrose in 1 liter of water, and subsequent adjustment of the pH. The sucrose is used to equalize the osmotic pressure of the buffer with that of the cells. The buffer is filtered with a 0.2-micron membrane and stored at 4° C. The concentrations of salts in the buffer as described result in a solution with electrical conductivity of approximately 0.014 S/m. The preferable range of the electrical conductivity of this buffer is between 1×10⁻³ and 2.5 S/m.

The pAcGFP-Cl plasmid (4.7 Kb, Clontech, Mountain View, Calif.) encodes a green fluorescent protein (GFP) from Aequorea coerulescens and contains an SV40 origin for replication in mammalian cells. The GFP protein is excited at 475 nm and emits at 505 nm. The plasmid is amplified in E. coli and purified using the QIAfilter Plasmid Mega Kit (Qiagen, Valencia, Calif.) according to the manufacturer's instructions. The plasmid DNA is dissolved in Tris-EDTA buffer and stored at −20° C. until use. The plasmid DNA concentration is determined by ultraviolet (UV) absorbance at 260 nm. Prior to an electroporation experiment, the plasmid is precipitated with ethanol and resuspended in phosphate buffered saline (PBS, 137 mM NaCl, 2.7 mM KCl, 10 mM Na₂HPO₄, 1.8 mM KH₂PO₄) buffer with an electrical conductivity of approximately 1.5 S/m at a concentration of approximately 40 ug/mL. The range of the electrical conductivity of this buffer is between 1×10⁻² and 10 S/m. The range of the plasmid concentration is between 0.01 and 100 ug/mL.

The low electrical conductivity buffer used for the cell flow inlet 105 (FIG. 2) used in combination with a higher electrical conductivity buffer (PBS) for the upper and lower sheath inlets 103 and 104 flow layers (FIG. 2) results in an electric field that is substantially larger across the cell flow layer for a given applied voltage. For a typical experiment, the pressure of each flow is adjusted so that the cell flow layer is approximately 50 microns deep and the upper and lower sheath flow layers are approximately 25 microns each in depth. The electrical conductivities of the low and high conductivity buffer are 0.014 S/m and 1.5 S/m, respectively. The electrical resistance of the sheath layer (for a voltage applied between the two support block surfaces 100 as shown in FIG. 2) is approximately 99% of the total resistance. This means that if 5 V is applied between the electrodes on the two support blocks that the electric fields in the streams adjacent to the electrodes is approximately 9 V/cm while the stream sandwiched between those two streams is 991 V/cm.

It is known that a difference of approximately 1 V between the interior and exterior of a certain cell will result in the formation of pores that can allow for the passage of nucleic acid molecules. The potential difference U across a cell membrane at a point on the surface of a cell in an external electric field of strength E is given by U=fER cos θ, where R is the cell radius, θ is the angle between the electric field and the normal to the cell surface, and f is a geometric factor that is typically around 3/2. This implies that to form pores at the poles of the cell the electric field should be about 1 kV/cm for a cell with radius of 8 microns.

With this electroporation device, the application of a 5 V potential difference between the top and bottom plates results in an electric field within the cell flow layer of about 1 kV/cm given the electric field strengths and flow layer depths described. The preferable range of applied voltages is between 1 V and 100 V. If the patterned electrodes are 2.5 cm by 0.5 cm in size, then for a 5 V applied potential, a current of about 0.17 A is generated and a power of 0.87 J/s is dissipated. This amount of power would increase the temperature of pure water in a device with dimensions 5 cm by 2.5 cm by 0.01 cm by 1.7 degrees C./s, assuming that no heat is dissipated through the boundary. The source for the applied voltage can be from a battery with a fixed voltage or a battery used in conjunction with a resistive voltage divider to enable the voltage to be varied over the selected range. Commercial voltage supplies are also readily available to provide selected voltages in the range of 1 V to 100 V. An alternative electrode size example includes electrodes with dimensions of 2.5 cm by 0.05 cm in size, then for a 5 V applied potential, a current of about 0.017 A is generated and a power of 0.087 J/s is dissipated. This amount of power would increase the temperature of pure water in a device with dimensions 5 cm by 2.5 cm by 0.01 cm by 0.17 degrees C./s. In a typical experiment, cells at a density of 1.0×10⁷/mL are flowed through the device (e.g., chip) at a volumetric rate of approximately 1.5 mL/min, with a preferable range between 0.01 and 100 mL/min. The nominal flow rate of 1.5 mL/min results in an average linear flow velocity of 1.0 cm/s. At this velocity, cells are subject to the electric field from an electrode that is 2.5 cm by 0.5 cm in width and length for 0.5 s. Assuming Hele-Shaw flow, the pressure difference across the input and output of the device (e.g., chip) is about 40 atm. It is important to note that during the approximately 0.5 s that cells are subject to the electric field, that the plasmid DNA is electrophoretically driven toward the cell flow layer, assuming that the plasmid is in the lower sheath flow and that the top electrode is held at a higher voltage than the bottom electrode. Assuming a DNA mobility of 4×10⁴ cm²/Vs, the average time that it takes a DNA molecule to move half-way through a distance of 25 microns (the typical depth of the sheath flow layer containing the plasmid) is 0.34 s. A DNA molecule that reaches the cell flow layer is driven across it in about 10 ms.

Another important timescale is the cell sedimentation time for falling a distance of one-half of the cell flow layer thickness. Again assuming a cell radius of 8 microns, a difference in density between a cell and the surrounding fluid of 0.07 g/cm³, and that the hydrodynamic friction coefficient of a cell is 6πηR, where η is the buffer viscosity (approximately 0.001 Pa, but may be higher with additive chemicals such as sucrose), the time to drop a distance of 25 microns is approximately 0.4 s. And the time for a typical salt ion, such as Na or K, to diffuse a distance of 25 microns is 0.6 s. This indicates that the flow layers remain laminar (and retain their respective conductivities) for the time it takes the cells to cross the electrode region when the patterned electrodes are about 2.5 cm by 0.5 cm in width and length.

Following the electroporation of a given volume of cells the electroporation efficiency and cell viability are determined by phase contrast and static fluorescent imaging, and sometimes by flow cytometry. After the cells are electroporated with the GFP-expressing plasmid in the flow device (e.g., chip), the cells are collected and transferred to a 96 or 24 well plate with appropriate cell medium, such as DMEM. The cells are cultured at 37° C. in an incubator with 5% CO₂ for 1, 6, 12, 24, or 48 hours. The cells are centrifuged at 300 g for 5 min and the aspirant is discarded. The cells are washed with PBS and this process is repeated. Following this, the cells are stained with propidium iodide (Invitrogen) at a concentration of approximately 1 microgram/mL. The cells are incubated in the dark for 15 min and then optically examined by phase contrast under fluorescent filters. A standard GFP filter set is used to determine the fraction of cells that have been electroporated with the plasmid. A filter set with excitation at 488 nm and emission at approximately 620 nm is used to determine the dead cells that have been permeated by propidium iodide. Several images can be acquired at different locations to improve the statistics of the electroporation efficiency and the cell viability. The cells may also be examined by flow cytometry to determine the fraction that has been electroporated as identified by a green fluorescent signal and the fraction that are dead as identified by uptake of propidium iodide and a red fluorescent signal.

Thus, the described device can reliably be used to electroporate a large number of mammalian or bacterial cells at high efficiency and with low cell death in a short amount of time. The cells can be transfected with plasmid DNA that can be transcribed into a protein that is therapeutic for a disease. The cells can be transfected with mRNA that is likewise transcribed into a protein that is necessary for improving the health of the cell or that can be harvested for other medical use, such as production of antibodies. The cells can also be transfected with purified Cas9 protein, or another DNA guided nuclease, and synthetic guide RNA molecules, termed ribonucleoproteins, to efficiently edit a genomic site that is deleterious.

### Example 2: Electroporation of Different Types of Cells and Molecules

The method outlined in Example 1 can be used to electroporate a variety of different mammalian cell types including: CHO, Hela, T-cells, CD8+, CD4+, CD3+, PBMC, Huh-7, Renca, NIH 3T3, Primary Fibroblasts, hMSCs, K562, Vero, HEK 293, A549, B16, BHK-21, C2C12, C6, CaCo-2, CAP-T, COS-1, Cos-7, CV-1, DLD-1, H1299, Hep G2, HOS, Jurkat, L5278Y, LNCaP, MCF7, MDA-MB-231, MDCK, Mesenchymal Stem Cells, Min-6, Neuro2a, NIH3T3L1, NSO, Panc-1, PC12, PC-3, RBL, RLE, SF21, SF9, SH-SY5Y, SK-MES-1, SK-N-SH, SL3, SW403, THP-1, U20S, and U937.

The method outlined in Example 1 can be used to electroporate a variety of different types of molecules to any mammalian cell including: DNA, RNA, mRNA, siRNA, miRNA, other nucleic acids, proteins, peptides, enzymes, metabolites, membrane impermeable drugs, cryoprotectants, exogenous organelles, molecular probes, nanoparticles, lipids, carbohydrates, small molecules, and complexes of proteins with nucleic acids (like CAS9-sgRNA). While the method outlined in Example 1 relies on an electric field to deliver charged nucleic acid molecules to electroporated cells, the method also suffices to electroporate neutral molecules where diffusive motion is sufficient for the delivery.

### Example 3: Electroporation Using Voltage Waveforms

The method outlined in Example 1 can be used to electroporate cells with a variety of different voltage waveforms applied. Cells can be electroporated when applying a square waveform with a frequency of 10 kHz and a peak to peak voltage difference of approximately 10 V. The preferable range of the peak to peak voltage difference is between 0.1 and 100 V. The preferable range of the frequency is between 100 Hz and 1 THz. The square wave could be bipolar so that the time averaged current is zero. The square wave could also have an additional DC component of preferably less than plus or minus 10 V. The applied waveform could be sinusoidal, saw-tooth, rectangular, triangular, or be a sum of any number of sinusoidal shapes with different frequencies and amplitudes in time.

The method outlined in Example 1 can be used to improve the efficiency of cellular electroporation by the application of different voltage waveforms to different electrode pairs. A first electrode pair could be used to apply a square waveform with a frequency of 10 kHz and a peak to peak voltage difference of approximately 10 V to permeabilize the membranes of the cells passing in the fluid channel. A second or third or more pair of electrodes could be used to apply a DC or oscillating voltage that preferentially directs charged molecules, like nucleic acids, toward the permeabilized cells. The second or third or more pair of electrodes could be used to exert electric forces on the cells or molecules in solution, which creates a relative velocity between the cells and the fluid, the molecules and the fluid, or between the cells and charged or neutral molecules in solution. The preferred range of the voltage amplitude or offset applied by the second or more pair of electrodes is between 1 mV and 100 V. The second or more pair of electrodes could be used to apply electrical forces that are along the direction of flow in the device or perpendicular to the direction of flow. Pairs of electrodes that are used to exert electric forces may be on the same surface or opposite surfaces of the device, where each surface is in contact with the fluid. The second pair of electrodes may be used to permeabilize structures within the cell after the membrane has been porated. The second or more pair of electrodes may be used to apply electric fields that result in the concentration increase of nucleic acid or other molecules at the interface between fluid layers of varying conductivity. The second or more pair of electrodes may be used to apply electric fields that cause the rotation of the cell so more surface area is exposed to the nucleic acid or other molecules in solution.

Although various embodiments have been depicted and described in detail herein, it will be apparent to those skilled in the relevant art that various modifications, additions, substitutions, and the like can be made without departing from the spirit of the disclosure and these are therefore considered to be within the scope of the disclosure as defined in the claims which follow.

### INCORPORATION BY REFERENCE

All U.S. patents, and U.S. and PCT patent application publications mentioned herein are hereby incorporated by reference in their entirety as if each individual patent or patent application publication was specifically and individually indicated to be incorporated by reference. In case of conflict, the present application, including any definitions herein, will control.

### EQUIVALENTS

Those skilled in the art will recognize or be able to ascertain using no more than routine experimentation many equivalents to the specific embodiments of the present invention described herein. Such equivalents are intended to be encompassed by the following claims.

1. An electroporation device comprising
a first-side support having at least one first-side electrode disposed at a region of an inner surface of the first-side support;
a second-side support having at least one second-side electrode disposed at a region of an inner surface of the second-side support, wherein the at least one second-side electrode is a counterpart electrode of the at least one first-side electrode; and
a fluid channel, having at least a portion of it between the first-side support and the second-side support, and having at least one fluid input and at least one fluid output, wherein the fluid channel allows fluid to flow continuously in at least one fluid stream toward the at least one fluid output,
wherein the at least one first-side electrode and the at least one second-side electrode are positioned in the electroporation device to allow modulating the electric field as a predetermined function of time and position in at least one part of the fluid channel between the first-side support and the second-side support.

2. The electroporation device of claim 1, further comprising at least one voltage supplier, wherein the at least one first-side electrode and said counterpart electrode of the at least one first-side electrode are connected to said voltage supplier independently from any other electrodes of the electroporation device, and wherein said voltage supplier allows modulating the electric field as a function of time in said at least one part of the fluid channel.

3. The electroporation device of claim 2, comprising a plurality of first-side electrodes comprising the at least one first-side electrode, and comprising a plurality of second-side electrodes comprising the at least one second-side electrode, wherein at least two sets of counterpart electrodes operate independently from each other.

4. The electroporation device of claim 3, wherein at least three sets of counterpart electrodes operate independently from each other.

5. The electroporation device of claim 3, further comprising one or more additional voltage suppliers, wherein each voltage supplier is connected to a different set of counterpart electrodes in the electroporation device.

6. The electroporation device of claim 5, wherein said voltage suppliers allow forming an electric field as a function of time and position within the fluid channel that maximizes an outcome function that positively correlates with cell transfection efficiency and negatively correlates with cell mortality.

7. The electroporation device of claim 5, wherein said voltage suppliers allow forming an electric field as a function of time and position within the fluid channel that maximizes an outcome function that positively correlates with electrode durability.

8. The electroporation device of claim 5, wherein said voltage suppliers allow independently controlling any two or more of the following: (a) opening pores in cells in a fluid in the fluid channel; (b) driving molecules into cells in a fluid in the fluid channel; (c) measuring an electrical property of a fluid in the fluid channel; (d) concentrating molecules at a part of the fluid channel; (e) moving cells in a fluid in the fluid channel to a part of the fluid channel; and (f) rotating cells in a fluid in the fluid channel.

9. The electroporation device of claim 2, wherein said voltage supplier provides a voltage that has a periodic waveform.

10-21. (canceled)

22. An electroporation device comprising
a first-side support having at least one first-side electrode disposed at a region of an inner surface of the first-side support;
a second-side support having at least one second-side electrode disposed at a region of an inner surface of the second-side support, wherein the at least one second-side electrode is a counterpart electrode of the at least one first-side electrode; and
a fluid channel, having at least a portion of it between the first-side support and the second-side support, and having at least two fluid inputs and at least one fluid output, wherein the fluid channel allows fluid to flow continuously in at least two fluid streams toward the at least one fluid output,
wherein the fluid channel allows modulating the flow rate, chemical composition, or both the flow rate and chemical composition in the at least two fluid streams as a predetermined function of time, position, or both time and position.

23. The electroporation device of claim 22, further comprising at least one fluid supplier, wherein said fluid supplier allows modulating the flow rate and chemical composition in one of the at least two streams as a predetermined function of time, position, or both time and position independently from any other fluid stream within the fluid channel.

24. The electroporation device of claim 23, further comprising one or more additional fluid suppliers, wherein each fluid supplier is connected to a different fluid input in the fluid channel.

25. The electroporation device of claim 24, wherein said fluid suppliers allow modulating the flow rate and chemical composition in separate fluid streams as a function of time and position within the fluid channel to maximize the time a cell in a fluid stream is in its optimal medium.

26. The electroporation device of claim 24, wherein said fluid suppliers allow modulating the flow rate and chemical composition in separate fluid streams as a function of time and position within the fluid channel to minimize the time a cell in a fluid stream is in an electroporation medium.

27. The electroporation device of claim 22, wherein the fluid channel has at least three fluid inputs allowing fluid to flow continuously in at least three fluid streams toward the at least one fluid output.

28-31. (canceled)

32. A system, comprising
an electroporation device, comprising
a first-side support having at least one first-side electrode disposed at a region of an inner surface of the first-side support;
a second-side support having at least one second-side electrode disposed at a region of an inner surface of the second-side support, wherein the at least one second-side electrode is a counterpart electrode of the at least one first-side electrode; and
a fluid channel, having at least a portion of it between the first-side support and the second-side support, and having at least one fluid input and at least one fluid output, wherein the fluid channel allows fluid to flow continuously in at least one fluid stream toward the at least one fluid output, wherein the at least one first-side electrode and the at least one second-side electrode are positioned in the electroporation device to allow modulating the electric field as a predetermined function of time and position in at least one part of the fluid channel between the first-side support and the second-side support; and
a fluid-delivery apparatus coupled to the electroporation device.

33-34. (canceled)

35. The system of claim 32, further comprising a fluid interface that couples the fluid-delivery apparatus to the electroporation device.

36-37. (canceled)

38. The system of claim 32, further comprising a cell processing module coupled to said electroporation device.

39. The system of claim 38, wherein said cell processing module is upstream from said electroporation device.

40-42. (canceled)

43. The system of claim 39, further comprising an apheresis bag upstream of the cell processing module.

44-57. (canceled)
